(19) United States
(12) Patent Application Publication
(10) Pub. No.: US 2024/0041755 A1
(43) Pub. Date: Feb. 8, 2024

(54) IMPLANTABLE CONSTRUCTS FOR MODULATING AN IMMUNE RESPONSE

(71) Applicant: William Marsh Rice University, Houston, TX (US)

(72) Inventors: Omid VEISEH, Houston, TX (US); Amanda NASH, Houston, TX (US); Boram KIM, Houston, TX (US); Damon BERMAN, Houston, TX (US); Lauren CHENG, Houston, TX (US); David ZHANG, Houston, TX (US)

(73) Assignee: William Marsh Rice University, Houston, TX (US)

(21) Appl. No.: 18/258,550

(22) PCT Filed: Dec. 17, 2021
(86) PCT No.: PCT/US2021/064149

§ 371 Date: Jun. 20, 2023

Related U.S. Application Data
(60) Provisional application No. 63/129,403, filed on Dec. 22, 2020.

(51) Int. Cl. A61K 9/00; A61K 47/36; A61K 9/50; A61K 35/12; C07K 14/52; A61K 39/00

(52) U.S. Cl. CPC A61K 9/0024; A61K 47/36; A61K 9/5036; A61K 35/12; C07K 14/52; A61K 39/0011

(57) ABSTRACT

The present disclosure relates to implantable constructs and related compositions comprising a plurality of cells producing antigens and/or immune effector molecules.

### CROSS REFERENCE TO RELATED APPLICATIONS

This application claims priority to U.S. Provisional Application No. 63/129,403, filed Dec. 22, 2020, which is hereby incorporated by reference in its entirety.

### BACKGROUND

Advances in biomedical research have led to methods for localized and targeted therapies for the treatment of diseases, such as cancer; however, in many instances, the percentage of patients responsive to these approaches remains modest (Park et al., Sci. Transl. Med. 10(433) 2018). In addition, many current therapies do not provide a means for controlling delivery of the therapeutic over time (i.e., downregulating or halting production). One approach to overcome these challenges entails use of implantable devices for delivery of therapeutic agents, which can provide local administration of a therapeutic agent in a host in a tunable manner. A need exists to identify implantable devices suitable for this purpose.

### SUMMARY

The present disclosure provides, at least in part, implantable constructs and related compositions, for example, comprising a plurality of cells producing antigens and/or immune effector molecules.

In some embodiments, a composition comprising a first implantable construct comprising an engineered cell that produces an antigen molecule; and a second implantable construct comprising an encapsulated engineered cell that produces an immune effector molecule are provided. In some embodiments, the antigen molecule induces an immune response in a subject, when, for example, the constructs are implanted into the subject.

In some embodiments, the antigen molecule comprises a nucleic acid, a protein, an antibody, antibody fragment, enzyme, cytokine, hormone, receptor, a lipid, a small molecule, a metabolic agent, an oligosaccharide, a peptide, or an amino acid. In some embodiments, the immune effector molecule activates an immune cell in a subject, represses an immune cell in a subject, and/or modulates immune cell migration in a subject. In some embodiments, the immune effector molecule enhances an immune response in a subject. In some embodiments, the immune effector molecule modulates host dendritic cell migration and/or host T cell activation. In some embodiments, the immune effector molecule enhances the immune response to the antigen molecule. In some embodiments, the immune effector molecule comprises a cytokine, such as but not limited to, IL-2, IL-12, IL-1, IL-1α, IL-1β, IL-1RA, IL-4, IL-5, IL-6, IL-7, IL-8, IL-9, IL-10, IL-11, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, and OSM.

In some embodiments, the first implantable construct, the second implantable construct, or both comprise an layer or zone encapsulating or admixed with the engineered cell of the first or second construct.

In some embodiments, compositions comprising a first implantable construct comprising an engineered cell that produces an antigen molecule; a second implantable construct comprising an encapsulated engineered cell that produces an immune effector molecule (e.g., a cytokine); and a third implantable construct comprising an engineered cell that produces an immune effector molecule (e.g., a different cytokine); wherein each implantable construct comprises, a layer, or zone encapsulating or admixed with the engineered cells or an inner zone and an outer zones, and wherein the layer, zone, or outer zone is configured so as to hinder contact of a host immune effector molecule or cell with the inner zone for an initial or shielded phase of implantation, but allows contact of a host immune effector molecule or cell with the inner zone in a subsequent or unshielded phase of implantation is provided.

In some embodiments, methods of forming a local or site-specific immune environment are provided, the methods comprising implanting into a subject, a composition or construct as provided for herein.

In some embodiments, methods of enhancing the immune response of a subject, are provided, wherein the methods comprise implanting into the subject a composition or construct as provided for herein.

In an embodiment, the composition of implantable constructs comprises a first cell that produces an antigen molecule and a second cell that produces an immune effector molecule. In an embodiment, each implantable construct in the composition comprises a single cell type. In an embodiment, each implantable construct in the composition comprises a plurality of cell types, e.g., a first cell that produces an antigen molecule and a second cell that produces an immune effector molecule. The implantable constructs described herein comprise a zone (e.g., a layer) to prevent contact of the antigen molecule and/or immune effector molecule from a host effector molecule, thereby shielding one or both from the host immune response. The layer or zone can be encapsulated or admixed with the engineered cell(s). In some embodiments, the layer or zone is free or substantially free of cells. In some embodiments, the outer layer comprises the engineered cells.

Without wishing to be bound by theory, the compositions comprising implantable constructs disclosed herein may act as a vaccine in a subject. In an embodiment, the composition comprising implantable constructs results in one or more of: (i) providing a controlled and sustained release of an antigen and immune effector molecule from the implantable construct; (ii) enabling local delivery of an antigen and immune effector molecule to yield systemic results; and (iii) modulating the activation and/or programming of a first host cell (e.g., a host effector T cell or a host NK cell) without activating or inducing expansion of a second host cell (e.g., a host T regulatory cell).

In an embodiment, the antigen molecule induces an immune response in a subject. In an embodiment, the antigen molecule comprises a nucleic acid (e.g., an RNA, a DNA, or an oligonucleotide), a protein (e.g., an antibody, antibody fragment, enzyme, cytokine, hormone, receptor), a lipid, a small molecule, a metabolic agent, an oligosaccharide, a peptide, or an amino acid. In an embodiment, the antigen comprises an exogenous antigen, endogenous antigen, autoantigen, neoantigen, viral antigen, or tumor antigen. In an embodiment, the first implantable construct provides sustained release of the antigen molecule. In an embodiment, the first implantable construct provides substantially non-pulsatile release of the antigen molecule. In an embodiment, the first implantable construct provides release of the antigen molecule for at least 1 day (e.g., longer than 2 days, 3 days, 4 days, 5 days, 6 days, 7 days, 8 days, 9 days, 10 days, 12 days, 14 days, 16 days, 18 days, or 20 days). In an embodiment, the first implantable construct provides release of the antigen molecule for at least 5 days (e.g., longer than 6 days, 7 days, 8 days, 9 days, 10 days, 12 days, 14 days, 16 days, 18 days, or 20 days).

In an embodiment, the immune effector molecule activates an immune cell, represses an immune cell, or modulates (e.g., initiates) immune cell migration. In an embodiment, the immune effector molecule enhances an immune response in a host organism. In an embodiment, the immune effector molecule comprises a cytokine. In an embodiment, the cytokine is selected from IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, and OSM. In an embodiment, the cytokine is selected from IL-2, IL-4, IL-7, IL-10, IL-12a, IL-12b, and IL-17. In some embodiments, the cytokine is IL-2. In some embodiments, the cytokine is IL-12 (e.g., IL-12a and/or IL-12b).

In another aspect, the present disclosure comprises a composition of implantable constructs that comprise: (i) a first cell that produces an antigen molecule; (ii) a second cell that produces a first immune effector molecule; (iii) and a third cell that produces a second immune effector molecule.

Certain embodiments described herein provide methods for the treatment or prevention of a disorder. In an embodiment, the disorder is a proliferative disorder or an infectious disease. These embodiments comprise administering to a subject an implantable construct described herein, e.g., comprising an antigen molecule and an immune effector molecule, for the treatment of the disease. The methods disclosed herein can result in a sustained release of a therapeutic agent at a target site (e.g., the intraperitoneal space), while having minimal or no effect at other non-target sites, thus avoiding the need for systemic delivery.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1A is an image depicting an exemplary embodiment of the disclosure. In this figure, three types of implantable constructs, each comprising a different engineered cell expressing either an antigen molecule or an immune effector molecule, work together to achieve activation of the host immune system. The composition is spatially and temporally regulated by the specific combination of antigen molecules and immune effector molecules expressed. The composition of implantable constructs may be customized by altering the antigen molecule, immune effector molecule, or the presence or ratio of either, which may allow for controlled and predictable modulation of the host immune system.

FIG. 1B is an image depicting an example of an implantable construct in which the construct degrades over time to expose the interior cells to the host immune system.

FIG. 2 is a graph showing production of each of the cytokines IL-2, IL-7, IL-10, and IL-12 in polymer-encapsulated retinal pigment epithelial (RPE) cells over time.

FIG. 3 is a schematic describing the integration of an exemplary gene insertion landing pad structure into the genome of a cell.

FIG. 4 is a schematic illustrating the components of an exemplary gene insertion landing pad structure, such as a constitutive promoter and a bicistronic open reading frame.

FIG. 5 is a schematic illustrating the integration of an exemplary gene of interest into the landing pad structure.

FIG. 6 is an image depicting an exemplary embodiment of the disclosure, namely the adaptive immunity induced in a host by a composition of implantable constructs implanted therein.

FIG. 7 is an imagine depicting exemplary vector maps for use in the methods described herein.

FIG. 8A illustrates survival curves for B16F10 mice threated with the encapsulated cells disclosed herein or sham surgery.

FIG. 8B illustrates tumor growth curves for B16F10 mice threated with the encapsulated cells disclosed herein or sham surgery.

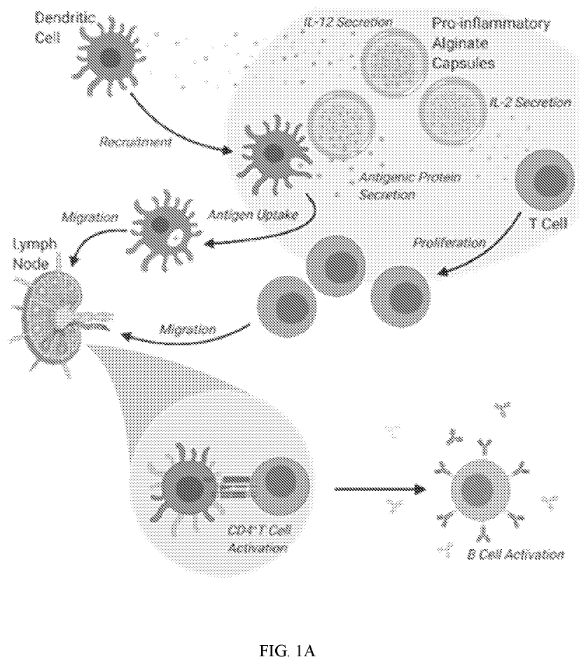

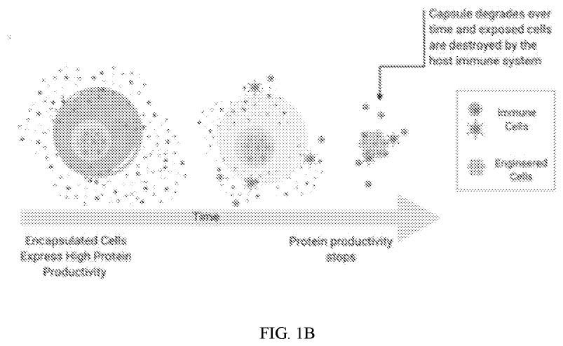

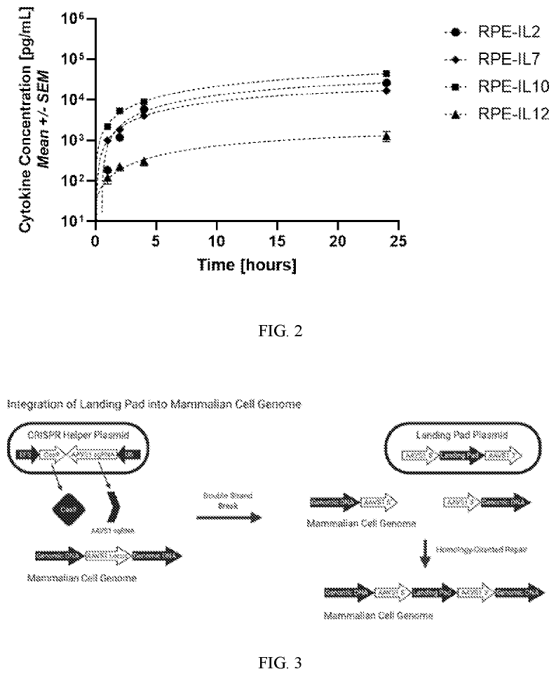

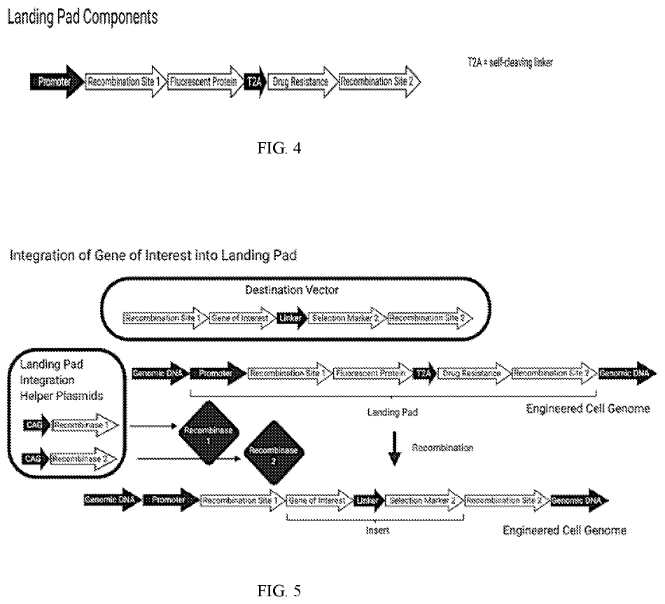

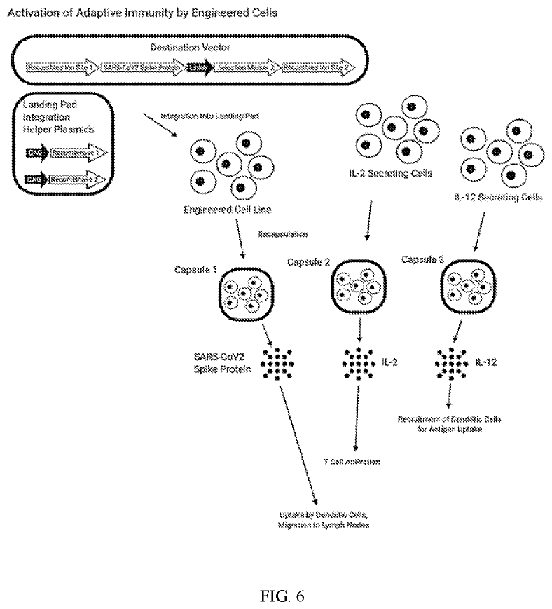

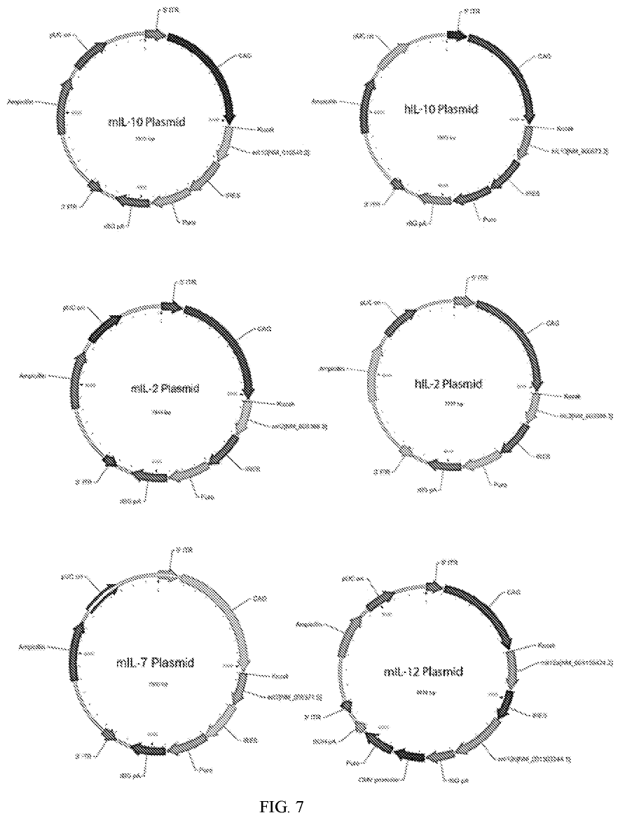

### DETAILED DESCRIPTION

The present disclosure features implantable constructs for delivery of an antigenic target (such as a cell and/or therapeutic agent) to a subject in a controlled release manner, and related methods of use thereof. The implantable constructs described herein comprise a zone or layer that encapsulates or admixes with the antigenic target, preventing contact of a host immune effector cell with the antigenic target to reduce immunoreactivity. In an embodiment, the zone is degradable, and allows for gradual removal of protection against the immune system in the case of encapsulated, therapy-producing cells or the gradual release of the antigenic target to the surrounding tissue or cells in the case where the antigenic target itself is intended for delivery. In some embodiments, the zone or layer is not degradable. In some embodiments, the zone or layer can become fibrosed or is not fibrosed when implanted into a subject (e.g., patient). The implantable constructs disclosed herein may comprise a single zone or a plurality of zones, may be formulated into different morphologies (e.g., spheres, rods, tubes), and may be prepared using a variety of materials. Each of these embodiments will be described below in more detail.

### I. Definitions

“Antigen molecule,” as used herein, is a substance which induces, activates, or evokes an immune response, e.g., in a subject.

“Cell,” as used herein, refers to an individual cell. In an embodiment, a cell is a primary cell or is derived from a cell culture. In an embodiment, a cell is a stem cell or is derived from a stem cell. A cell may be xenogeneic, autologous, or allogeneic. In an embodiment, a cell is be engineered (e.g., genetically engineered) or is not engineered (e.g., not genetically engineered).

“Degradable,” as used herein, refers to a structure which upon modulation, e.g., cleavage, decreases the ability of the zone of the implantable construct (e.g., the inner zone and/or the outer zone) to impede contact of a host immune effector molecule with the zone (e.g., the inner zone and/or the outer zone) or a component disposed in the zone. For example, the degradable entity can comprise a site which is cleavable by an enzyme, e.g., an endogenous host enzyme, or an administered enzyme. Typically, the degradable entity mediates a physical property of a zone, e.g., the inner zone or the outer zone, for example, the thickness, degree of cross-linking, or permeability, which impedes passage of a host agent (e.g., a host immune component, e.g., a host immune cell).

“Immune effector molecule,” as used herein, is a substance which interacts with or regulates an immune response in a subject (e.g., a host). An immune effector molecule may activate the immune response in a subject, repress the immune response in a subject, or modulate (e.g., initiate) immune cell migration in a subject. In an embodiment, the immune effector molecule activates or represses an immune cell in a subject as described herein. Exemplary immune effector molecules include cytokines, such as IL-2, IL-7, IL-10, and IL-12.

“Prevention,” “prevent,” and “preventing” as used herein refers to a treatment that comprises administering or applying a therapy, e.g., administering an implantable construct (e.g., as described herein) comprising an antigen molecule or an immune effector molecule prior to the onset of a disease or condition in order to preclude the physical manifestation of said disease or condition. In some embodiments, “prevention,” “prevent,” and “preventing” require that signs or symptoms of the disease or condition have not yet developed or have not yet been observed. In some embodiments, treatment comprises prevention and in other embodiments it does not.

“Subject,” as used herein, refers to the recipient of the implantable construct described herein. The subject may include a human and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development (e.g., a male or female of any age group, e.g., a pediatric subject (e.g., infant, child, adolescent) or adult subject (e.g., young adult, middle-aged adult, or senior adult). A non-human animal may be a transgenic animal.

“Treatment,” “treat,” and “treating,” as used herein, refer to reversing, alleviating, delaying the onset of, or inhibiting the progress of one or more of a symptom, manifestation, or underlying cause of a disease or condition. (e.g., as described herein), e.g., by administering or applying a therapy, e.g., administering an implantable construct comprising an antigen molecule or immune effector molecule. In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease or condition. In an embodiment, treating comprises reducing, reversing, alleviating, reducing, or delaying the onset of, an underlying cause of a disease or condition. In some embodiments, “treatment,” “treat,” and “treating” require that signs or symptoms of the disease or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

Selected Chemical Definitions. Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present disclosure.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁_C₁₀ alkyl (e.g., —CH₃). In certain embodiments, the alkyl group is substituted C₁_C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₅ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents, e.g., for instance from 1 to 5 substituents, 1 to 3 substituents, or 1 substituent. In certain embodiments, the alkenyl group is unsubstituted C₁_C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₅ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₅ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₅ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₈), bicyclo[2.2.2]octanyl (C₈), bicyclo[2.1.1]hexanyl (C₆), bicyclo[3.1.1]heptanyl (C₇), bicyclo[2.2.1]hept-2-enyl (C₇) (norbornenyl), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like.

The terms “alkylene,” “alkenylene,” “alkynylene,” “haloalkylene,” “heteroalkylene,” “cycloalkylene,” “heterocyclylene,” “arylene”, or “heteroarylene” alone or as part of another substituent, mean, unless otherwise stated, a divalent radical derived from an alkyl, alkenyl, alkynyl, haloalkylene, heteroalkylene, cycloalkyl, heterocyclyl, aryl, or heteroaryl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, “oxo” refers to a carbonyl, i.e., —C(O)—.

The symbol “ [symbol] ” as used herein in relation to a compound, e.g., a compound of Formula (I), refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E.L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compounds described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

The term “pharmaceutically acceptable salt” is meant to include salts of the compounds described herein that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for the present disclosure.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of the present disclosure may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### A. IMPLANTABLE CONSTRUCTS

An implantable construct described herein comprises a material that reduces or inhibits a reaction (e.g., such as an immunomodulatory reaction) with or on an antigen molecule or immune effector molecule disposed within. For example, an implantable construct comprises a zone or layer that shields an antigen molecule or immune effector molecule from exposure to the surrounding milieu, such as host tissue, host cells, or host cell products. In an embodiment, an implantable construct minimizes the effect of a host response (e.g., an immune response) directed at an antigen molecule or immune effector molecule disposed within, e.g., as compared with a similar antigen molecule or immune effector molecule that is not disposed within an implantable construct.

The implantable construct may comprise a permeable, semi-permeable, or impermeable material to control the flow of solution in and out of the implantable construct. For example, the material may be permeable or semi-permeable to allow free passage of small molecules, such as nutrients and waste products, in and out of the construct. In addition, the material may be permeable or semi-permeable to allow the transport of an antigen molecule or immune effector molecule out of the implantable construct. Exemplary materials include polymers, metals, ceramics, and combinations thereof.

In an embodiment, the implantable construct comprises a polymer (e.g., a naturally occurring polymer or a synthetic polymer). For example, a polymer may comprise polystyrene, polyester, polycarbonate, polyethylene, polypropylene, polyfluorocarbon, nylon, polyacetylene, polyvinyl chloride (PVC), polyolefin, polyurethane, polyacrylate, polymethacrylate, polyacrylamide, polymethacrylamide, polymethyl methacrylate, poly(2-hydroxyethyl methacrylate), polysiloxane, polydimethylsiloxane (PDMS), polyhydroxyalkanoate, PEEK®, polytetrafluoroethylene, polyethylene glycol, polysulfone, polyacrylonitrile, collagen, cellulose, cellulosic polymers, polysaccharides, polyglycolic acid, poly(L-lactic acid) (PLLA), poly(lactic glycolic acid) (PLGA), polydioxanone (PDA), poly(lactic acid), hyaluronic acid, agarose, alginate, chitosan, or a blend or copolymer thereof. In an embodiment, the implantable construct comprises a polysaccharide (e.g., alginate, cellulose, hyaluronic acid, or chitosan). In an embodiment, the implantable construct comprises alginate. In some embodiments, the average molecular weight of the polymer is from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). The implantable construct may comprise at least 0.5%, 1%, 2%, 3%, 4%, 5%, 7.5%, 10%, 15%, 20%, 30%, 40%, 50%, 60%, 70%, 80% or more of a polymer, e.g., a polymer described herein.

In an embodiment, the implantable construct comprises a polysaccharide, e.g., an alginate. Alginate is a naturally occurring polymer comprising 0-(1-4)-linked mannuronic acid and guluronic acid residues, and as a result of its high density of negatively charged carboxylates, may be cross-linked with certain cations to form a larger structure, such as a hydrogel. Alginate polymers described herein may have an average molecular weight from about 2 kDa to about 500 kDa (e.g., from about 2.5 kDa to about 175 kDa, from about 5 kDa about 150 kDa, from about 10 kDa to about 125 kDa, from about 12.5 kDa to about 100 kDa, from about 15 kDa to about 90 kDa, from about 17.5 kDa to about about 80 kDa, from about 20 kDa to about 70 kDa, from about 22.5 kDa to about 60 kDa, or from about 25 kDa to about 50 kDa). In an embodiment, the implantable construct comprises at least 0.5%, 1%, 2%, 3%, 4%, 5%, 7.5%, 10%, 15%, 20%, 30%, 40%, 50%, 60%, 70%, 80% or more of an alginate polymer. In an embodiment, the alginate is an ultrapure alginate (e.g., SLG20 alginate).

In an embodiment, the implantable construct comprises a metal or a metallic alloy. Exemplary metals or metallic alloys include titanium (e.g., nitinol, nickel titanium alloys, thermo-memory alloy materials), platinum, platinum group alloys, stainless steel, tantalum, palladium, zirconium, niobium, molybdenum, nickel-chrome, cobalt, tantalum, chromium molybdenum alloys, nickel-titanium alloys, and cobalt chromium alloys. In an embodiment, the implantable construct comprises stainless steel grade. The implantable construct may comprise at least 0.5%, 1%, 2%, 3%, 4%, 5%, 7.5%, 10%, 15%, 20%, 30%, 40%, 50%, 60%, 70%, 80% or more of a metal or metallic alloy, e.g., a metal or metallic alloy described herein.

In an embodiment, the implantable construct comprises a ceramic. Exemplary ceramics include a carbide, nitride, silica, or oxide materials (e.g., titanium oxides, hafnium oxides, iridium oxides, chromium oxides, aluminum oxides, and zirconium oxides). The implantable construct may comprise at least 0.5%, 1%, 2%, 3%, 4%, 5%, 7.5%, 10%, 15%, 20%, 30%, 40%, 50%, 60%, 70%, 80% or more of a ceramic, e.g., a ceramic described herein.

In an embodiment, the implantable construct may comprise glass. The implantable construct may comprise at least 0.5%, 1%, 2%, 3%, 4%, 5%, 7.5%, 10%, 15%, 20%, 30%, 40%, 50%, 60%, 70%, 80% or more glass.

A material within an implantable construct may be further modified, for example, with a chemical modification. For example, a material may be coated or derivatized with a chemical modification that provides a specific feature, such as an immunomodulatory or antifibrotic feature. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein.

### B. CELLS

Implantable constructs described herein may contain a cell, for example, an engineered cell. A cell be derived from any mammalian organ or tissue, including the brain, nerves, ganglia, spine, eye, heart, liver, kidney, lung, spleen, bone, thymus, lymphatic system, skin, muscle, pancreas, stomach, intestine, blood, ovary, uterus, or testes.

A cell may be derived from a donor (e.g., an allogeneic cell), derived from a subject (e.g., an autologous cell), or from another species (e.g., a xenogeneic cell). In an embodiment, a cell can be grown in cell culture, or prepared from an established cell culture line, or derived from a donor (e.g., a living donor or a cadaver). In an embodiment, a cell is genetically engineered. In another embodiment, a cell is not genetically engineered. A cell may include a stem cell, such as a reprogrammed stem cell, or an induced pluripotent cell. Exemplary cells include mesenchymal stem cells (MSCs), fibroblasts (e.g., primary fibroblasts). HEK cells (e.g., HEK293T), Jurkat cells, HeLa cells, retinal pigment epithelial (RPE) cells, HUVEC cells, NIH3T3 cells, CHO-K1 cells, COS-1 cells, COS-7 cells, PC-3 cells, HCT 116 cells, A549MCF-7 cells, HuH-7 cells, U-2 OS cells, HepG2 cells, Neuro-2a cells, and SF9 cells. In an embodiment, a cell for use in an implantable construct is an RPE cell.

A cell included in an implantable construct may produce or secrete antigen molecule and/or an immune effector molecule. In an embodiment, a cell included in an implantable construct may produce or secrete a single type of antigen molecule or a plurality of antigen molecules. In an embodiment, a cell included in an implantable construct may produce or secrete a single type of immune effector molecule or a plurality of immune effector molecule. In an embodiment, an implantable construct may comprise a cell that is transduced or transfected with a nucleic acid (e.g., a vector) comprising an expression sequence of an antigen molecule or an immune effector molecule. For example, a cell may be transduced or transfected with a lentivirus. A nucleic acid introduced into a cell (e.g., by transduction or transfection) may be incorporated into a nucleic acid delivery system, such as a plasmid, or may be delivered directly. In an embodiment, a nucleic acid introduced into a cell (e.g., as part of a plasmid) may include a region to enhance expression of the antigen molecule or immune effector molecule and/or to direct targeting or secretion, for example, a promoter sequence, an activator sequence, or a cell-signaling peptide, or a cell export peptide. Exemplary promoters include EF-1a, CMV, Ubc, hPGK, VMD2, and CAG. Exemplary activators include the TET1 catalytic domain, P300 core, VPR, rTETR, Cas9 (e.g., from S. pyogenes or S. aureus), and Cpf1 (e.g., from L. bacterium).

An implantable construct described herein may comprise a cell or a plurality of cells. In the case of a plurality of cells, the concentration and total cell number may be varied depending on a number of factors, such as cell type, implantation location, and expected lifetime of the implantable construct. In an embodiment, the total number of cells included in an implantable construct is greater than about 2, 4, 6, 8, 10, 20, 30, 40, 50, 75, 100, 200, 250, 500, 750, 1000, 1500, 2000, 5000, 10000, or more. In an embodiment, the total number of cells included in an implantable construct is greater than about 1.0×10², 1.0×10³, 1.0×10⁴, 1.0×10⁵, 1.0×10⁶, 1.0×10⁷, 1.0×10⁸, 1.0×10⁹, 1.0×10¹⁰, or more. In an embodiment, the total number of cells included in an implantable construct is less than about than about 10000, 5000, 2500, 2000, 1500, 1000, 750, 500, 250, 200, 100, 75, 50, 40, 30, 20, 10, 8, 6, 4, 2, or less. In an embodiment, the total number of cells included in an implantable construct is less than about 1.0×10¹⁰, 1.0×10⁹, 1.0×10⁸, 1.0×10⁷, 1.0×10⁶, 1.0×10⁵, 1.0×10⁴, 1.0×10³, 1.0×10², or less. In an embodiment, a plurality of cells is present as an aggregate. In an embodiment, a plurality of cells is present as a cell dispersion.

Specific features of a cell contained within an implantable construct may be determined, e.g., prior to and/or after incorporation into the implantable construct. For example, cell viability, cell density, or cell expression level may be assessed. In an embodiment, cell viability, cell density, and cell expression level may be determined using standard techniques, such as cell microscopy, fluorescence microscopy, histology, or biochemical assay.

### C. ANTIGEN MOLECULES AND IMMUNE EFFECTOR MOLECULES

The compositions described herein comprise implantable constructs encapsulating cells that contain either an antigen molecule, an immune effector cell, or both. In an embodiment, the antigen molecule or immune effector molecule is produced (e.g., expressed or secreted) by a cell. The cell can be within or admixed within the implantable construct, such as admixed or encapsulated by a polymer as provided for herein. In some embodiments, the polymer is an alginate, such as, but not limited to, those described herein.

An antigen molecule provided in the implantable constructs described herein may include a nucleic acid (e.g., an RNA, a DNA, or an oligonucleotide), a protein (e.g., an antibody, antibody fragment, enzyme, cytokine, hormone, receptor), a lipid, a small molecule, a metabolic agent, an oligosaccharide, a peptide, or an amino acid. In an embodiment, the implantable construct comprises a cell or a plurality of cells that are genetically engineered to produce (e.g., express or secrete) an antigen molecule. In some embodiments, the molecule is a cytokine. In some embodiments, the cytokine is IL-2 or IL-12 (IL-12a and/or IL-12b). These can be co-expressed with an antigen from the implantable constructs, which can be the same or different as provided for herein, to enhance an immune response against the antigen. In some embodiments, the antigen is a tumor antigen.

The antigen molecule may comprise a molecule present on the surface of a pathogen or cell. For example, the antigen molecule may comprise a cell surface molecule (e.g., a glycoprotein). In an embodiment, the antigen is capable of binding to an antigen-specific antibody or B-cell antigen receptor. An antigen may be an exogenous antigen, endogenous antigen, autoantigen, neoantigen, viral antigen, or tumor antigen. In some embodiments, the antigen is a tumor antigen. In some embodiment, the antigen is a melanoma antigen or a pancreatic tumor antigen.

In an embodiment, the antigen molecule is a peptide. The peptide may comprise two or more amino acid residues, e.g., at least 2, 3, 4, 5, 6, 7, 8, 9, 10, 11, 12, 13, 14, 15, 16, 17, 18, 19, 20, 22, 24, 26, 28, 30, 32, 34, 36, 38, 40, 42, 44, 46, 48, or 50 amino acid residues. In an embodiment, the peptide is a linear peptide or a cyclic peptide. In an embodiment, the peptide may be modified, e.g., by glycosylation, methylation, or other known natural or synthetic modification. A peptide may be produced or secreted as a pre-peptide or in an inactive form and may require further modification to convert it into an active form.

In an embodiment, the antigen molecule is a protein. The protein may be of any size, e.g., greater than about 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the protein is composed of a single subunit or multiple subunits (e.g., a dimer, trimer, tetramer, etc.). A protein antigen produced or secreted by a cell may be modified, for example, by glycosylation, methylation, or other known natural or synthetic protein modification. A protein antigen may be produced or secreted as a pre-protein or in an inactive form and may require further modification to convert it into an active form.

Proteins produced or secreted by a cell may be include antibodies or antibody fragments, for example, an Fc region or variable region of an antibody. Exemplary antibodies include anti-PD-1, anti-PD-L1, anti-CTLA4, anti-TNFα, and anti-VEGF antibodies. An antibody may be monoclonal or polyclonal. Other exemplary proteins include a lipoprotein, an adhesion protein, blood clotting factor (e.g., Factor VII, Factor VIII, Factor IX, GCG, or VWF), hemoglobin, enzymes, proenkephalin, a growth factor (e.g., EGF, IGF-1, VEGF alpha, HGF, TGF beta, bFGF), or a cytokine.

An antigen molecule described herein may include a hormone. Exemplary hormones include growth hormone, growth hormone releasing hormone, prolactin, luteinizing hormone (LH), anti-diuretic hormone (ADH), oxytocin, thyroid stimulating hormone (TSH), thyrotropin-release hormone (TRH), adrenocorticotropic hormone (ACTH), follicle-stimulating hormone (FSH), thyroxine, calcitonin, parathyroid hormone, aldosterone, cortisol, epinephrine, glucagon, insulin, estrogen, progesterone, and testosterone.

An antigen molecule described herein may be a viral antigen. In an embodiment, a viral antigen is any molecule derived from a virus, such a capsid protein, a spike protein, or a fragment thereof. Exemplary viral antigens may be derived from a viral family including Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae, Corticoviridae, Cystoviridae, Deltavirus, Dianthovirus, Enamovirus, Filoviridae Flaviviridae, Hepadnaviridae, Herpesviridae, Hypoviridae, Iridoviridae, Leviviridae, Lipothrixviridae, Microviridae, Orthomyxoviridae, Papovaviridae, Paramyxoviridae, Parvoviridae, Picornaviridae, Poxviridae, Reoviridae, Retroviridae, Rhabdoviridae, Togaviridae, and Totiviridae. A viral antigen described herein may be derived from marburg virus, ebola virus, dengue virus, rabies virus, rotavirus, rubella virus, measles virus, respiratory syncytial virus, hepatitis A virus, hepatitis B virus, hepatitis C virus, hepatitis D virus, herpes virus, human immunodeficiency virus (HIV), rhinovirus, vaccinia virus, norovirus, Epstein-Barr virus, Rift Valley fever virus, West Nile virus, Hantaa virus, human papillomavirus, smallpox virus, poliovirus, rhinovirus, hepatovirus, aphthovirus, measles, mumps, influenza, Norwalk virus, Zika virus, Japanese encephalitis virus, yellow fever virus, simian foamy virus, western equine encephalitis virus, variola virus, severe acute respiratory syndrome coronavirus (SARS CoV), severe acute respiratory syndrome coronavirus 2 (SARS CoV-2), Middle East respiratory syndrome virus, or any strain thereof. In an embodiment, the viral antigen is derived from severe acute respiratory syndrome coronavirus 2 (SARS CoV-2).

An antigen molecule described herein may be a bacterial antigen. In an embodiment, a bacterial antigen is any molecule derived from a bacterium, such lipopolysaccharide, endotoxin, a bacterial protein, or a fragment thereof. Exemplary bacterial antigens may be derived from a bacterial family including Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus, Hyphomicrobium, Legionella, Leptspirosis, Listeria, Meningococcus, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

An antigen molecule described herein may be a tumor antigen. In an embodiment, a tumor antigen is a molecule present on or in the surface of a tumor cell. A tumor antigen may differ from a non-antigen present in a subject by a single point mutation or several mutations (e.g., a tumor-specific antigen). Exemplary tumor antigens include alpha-actinin-4, Bcr-Abl, Casp-8, beta-catenin, cdc27, cdk4, cdkn2a, coa-1, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pm1-RARa fusion protein, PTPRK, K-ras, N-ras, Triosephosphate isomerase, Bage-1, Gage 3,4,5,6,7, GnTV, Herv-K-mel, Lage-1, Mage-A1,2,3,4,6,10,12, Mage-C₂, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, b-Catenin, CDK4, Mum-1, p16, TAGE, PSMA, PSCA, CT7, telomerase, 43-9F, 5T4, 791Tgp72, a-fetoprotein, 13HCG, BCA225, BTAA, CA 125, CA 15-3 (CA 27.29BCAA), CA 195, CA 242, CA-50, CAM43, CD68KP1, CO-029, FGF-5, G250, Ga733 (EpCAM), HTgp-175, M344, MA-50, MG7-Ag, MOV18, NB70K, NY-CO-1, RCAS1, SDCCAG16, TA-90 (Mac-2 binding protein\cyclophilin C-associated protein), TAAL6, TAG72, TLP, and TPS, or a fragment thereof. A tumor antigen may be associated with any cancer, for example, a cancer described on page 20 herein.

An antigen molecule described herein may be selected using an antigen selection technique, e.g., an in silico antigen selection process. For example, an antigen described herein may be selected using an automated or semi-automated in silico process for identifying tumor antigens from tumor mutation and expression data, e.g., to identify antigens predicted to bind and be presented by HLA class I molecules and elicit anti-tumor T cell immunity, e.g., in a subject. In some embodiments, the in silico antigen selection process involves the analysis of massively parallel DNA and RNA sequencing data, to systematically identify and shortlist candidate antigen peptides from a tumor's mutational repertoire that could potentially be used in a vaccine.

In some embodiments, the in silico antigen selection process involves the steps of: obtaining a list of non-synonymous mutations identified by a somatic variant-calling pipeline, using exomic sequencing and transcript sequencing of both normal and tumor tissue; and annotating the list with amino acid changes (e.g., amino acid changes arising from a missense mutation) and transcript sequences. Amino acid FASTA sequences comprising mutated and wild-type sequences (e.g., 17-21-mer sequences) may then be built and input into the in silico process, together with HLA haplotype data, e.g., obtained from the subject to be treated, provided by using clinical genotyping assays or in silico approaches. Subsequent analysis in silico can perform epitope prediction, integrate sequencing-based information, and filter antigen candidates (e.g., limiting to peptide sequences predicted to have the strongest binding (e.g., a mutant (MT) binding score of less than 500 nM, e.g., less than 250 nM); eliminating wild-type (WT) peptides; and/or limiting to peptides that are expressed as an RNA variant), thereby identifying tumor antigens predicted to bind and be presented by HLA class I molecules and elicit anti-tumor T cell immunity.

In some embodiments, the antigen molecule is selected using a personalized Variant Antigens by Cancer Sequencing (pVAC-Seq) method. In some embodiments, the antigen molecule is selected using a method described in Hundal, et al. (Genome Med. (2016) 8:11) or Carreno et al (U.S. Patent Publication No. 2017/0202939), each of which are incorporated herein by reference in their entirety. Antigens selected by an antigen selection technique may be further validated using biochemical or cellular assays, and/or incorporated into a vaccine or formulation thereof, using known methods, e.g., as described by Carreno (vide supra).

An antigen molecule described herein may be an autoantigen. In an embodiment, an autoantigen is a protein or nucleic acid derived from a subject that is recognized by the immune system of the subject. In an embodiment, the autoantigen is a self-antigen. In an embodiment, the autoantigen is not tissue specific. In an embodiment, the autoantigen is a tRNA synthetase. Exemplary autoantigens are those that are associated with a disease including celiac disease, lupus erythematosus, rheumatoid arthritis, dermatomyostis, scleroderma, sarcoidosis, vitiligo, multiple sclerosis, gluten ataxia, autoimmune encephalitis, idiopathic thrombocytopenic purpura, Crohn's disease, Hashimoto's thyroiditis, Addison's disease, diabetes mellitus type 1, pemphigus vulgaris, pernicious vulgaris, and autoimmune hemolytic anemia. In an embodiment, an autoantigen is capable of recognition by an autoantibody.

An antigen molecule described herein may be an allergen, such an environmental allergen. For example, an antigen may be a molecule produced by a non-host cell, such as a plant, bacterial, fungal, or insect cell. An allergen may be a pollen allergen (e.g., from a tree or grass), an animal allergen (e.g., animal hair or dander), insect allergen (e.g., venom), food allergen (e.g., peanut allergen, wheat allergen, gluten allergen) or a fragment thereof. The allergen may be naturally occurring or produced by humans, e.g., a detergent, household chemical, pesticide, dye, or pharmaceutical.

In an embodiment, an implantable construct comprises a cell expressing a single type of antigen molecule or may express more than one type of antigen molecule, e.g., a plurality of antigen molecules. In an embodiment, an implantable construct comprises a cell expressing two types of antigen molecules. In an embodiment, an implantable construct comprises a cell expressing three types of antigen molecules. In an embodiment, an implantable construct comprises a cell expressing four types of antigen molecules.

The immune effector cell described herein may activate an immune cell, repress an immune cell, and/or modulates (e.g., initiate) immune cell migration, e.g., in a subject. In an embodiment, the immune effector molecule modulates host dendritic cell migration or host cell T activation.

An immune effector molecule provided in the implantable constructs described herein may include a cytokine. A cytokine may be apro-inflammatory cytokine, an anti-inflammatory cytokine, or a chemokine (e.g., which may initiate immune cell migration). Example of cytokines include IL-1, IL-1α, IL-1β, IL-1RA, IL-2, IL-4, IL-5, IL-6, IL-7, IL-8, IL-9, IL-10, IL-11, IL-12, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, OSM, and others. For example, a cytokine may include any cytokine described in M. J. Cameron and D. J. Kelvin, Cytokines, Chemokines, and Their Receptors (2013), Landes Biosciences, which is incorporated herein by reference in its entirety.

In an embodiment, the immune effector molecule (e.g., a cytokine) is a protein. The immune effector molecule (e.g., cytokine) may be of any size, e.g., greater than about 50 Da, 100 Da, 200 Da, 250 Da, 500 Da, 750 Da, 1 KDa, 1.5 kDa, 2 kDa, 2.5 kDa, 3 kDa, 4 kDa, 5 kDa, 6 kDa, 7 kDa, 8 kDa, 9 kDa, 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 95 kDa, 100 kDa, 125 kDa, 150 kDa, 200 kDa, 200 kDa, 250 kDa, 300 kDa, 400 kDa, 500 kDa, 600 kDa, 700 kDa, 800 Da, 900 kDa, or more. In an embodiment, the immune effector molecule (e.g., cytokine) is composed of a single subunit or multiple subunits (e.g., a dimer, trimer, tetramer, etc.). An immune effector molecule produced or secreted by a cell may be modified, for example, by glycosylation, methylation, or other known natural or synthetic protein modification. A immune effector molecule may be produced or secreted as a pre-protein or in an inactive form and may require further modification to convert it into an active form.

An implantable construct may comprise a cell expressing a single type of immune effector molecule (e.g., single type of cytokine), or may express more than one type of immune effector molecule (e.g., a plurality of cytokines). In an embodiment, an implantable construct comprises a cell expressing two types of immune effector molecules (e.g., two types of cytokines). In an embodiment, an implantable construct comprises a cell expressing three types of immune effector molecules (e.g., three types of cytokines). In an embodiment, an implantable construct comprises a cell expressing four types of immune effector molecules (e.g., four types of cytokines).

The implantable elements found within the compositions described herein may work together to modulate the immune response in a subject, e.g., in the manner of a vaccine. In an embodiment, after administration of the compositions described herein to a subject, subsequent exposure of the subject to the antigen or a related molecule results in an improved outcome in the subject. For example, upon subsequence exposure of the subject to the antigen or a related molecule, the subject may experience one or more of: (i) reduced severity of a symptom of a disease, disorder, or condition compared with a subject not previously exposed to the disclosed compositions; (ii) a shorter duration of a symptom of disease, disorder, or condition compared with a subject not previously exposed to the disclosed compositions; (iii) faster production of antibodies targeting the antigen molecule or a related molecule in the disclosed compositions compared with a subject not previously exposed to the disclosed compositions.

In some embodiments, the immune effector molecule and/or antigen has a sequence recited in Table 1.

TABLE 1

| Cytokine Name | Sequences used |
|---|---|
| mIL-2 | ATGTACAGCATGCAGCTCGCATCCTGTGTCACATTGACACTTGTGCTCCTTGTCAACAGC |
| SEQ ID NO: 1 | GCACCCACTTCAAGCTCCACTTCAAGCTCTACAGCGGAAGCACAGCAGCAGCAGCAGCA |
| | GCAGCAGCAGCAGCAGCAGCACCTGGAGCAGCTGTTGATGGACCTACAGGAGCTCCTG |
| | AGCAGGATGGAGAATTACAGGAACCTGAAACTCCCCAGGATGCTCACCTTCAAATTTTA |
| | CTTGCCCAAGCAGGCCACAGAATTGAAAGATCTTCAGTGCCTAGAAGATGAACTTGGAC |
| | CTCTGCGGCATGTTCTGGATTTGACTCAAAGCAAAAGCTTTCAATTGGAAGATGCTGAG |
| | AATTTCATCAGCAATATCAGAGTAACTGTTGTAAAACTAAAGGGCTCTGACAACACATTT |
| | GAGTGCCAATTCGATGATGAGTCAGCAACTGTGGTGGACTTTCTGAGGAGATGGATAG |
| | CCTTCTGTCAAAGCATCATCTCAACAAGCCCTCAATAA |
| hIL-2 | ATGTACAGGATGCAACTCCTGTCTTGCATTGCACTAAGTCTTGCACTTGTCACAAACAGT |
| SEQ ID NO: 2 | GCACCTACTTCAAGTTCTACAAAGAAAACACAGCTACAACTGGAGCATTTACTGCTGGAT |
| | TTACAGATGATTTTGAATGGAATTAATAATTACAAGAATCCCAAACTCACCAGGATGCTC |
| | ACATTTAAGTTTTACATGCCCAAGAAGGCCACAGAACTGAAACATCTTCAGTGTCTAGAA |
| | GAAGAACTCAAACCTCTGGAGGAAGTGCTAAATTTAGCTCAAAGCAAAAACTTTCACTT |
| | AAGACCCAGGGACTTAATCAGCAATATCAACGTAATAGTTCTGGAACTAAAGGGATCTG |
| | AAACAACATTCATGTGTGAATATGCTGATGAGACAGCAACCATTGTAGAATTTCTGAAC |
| | AGATGGATTACCTTTTGTCAAAGCATCATCTCAACACTGACTTGA |
| mIL-7 | ATGTTCCATGTTTCTTTTAGATATATCTTTGGAATTCCTCCACTGATCCTTGTTCTGCTGCC |
| SEQ ID NO: 3 | TGTCACATCATCTGAGTGCCACATTAAAGACAAAGAAGGTAAAGCATATGAGAGTGTAC |
| | TGATGATCAGCATCGATGAATTGGACAAAATGACAGGAACTGATAGTAATTGCCCGAAT |
| | AATGAACCAAACTTTTTTAGAAAACATGTATGTGATGATACAAAGGAAGCTGCTTTTCTA |
| | AATCGTGCTGCTCGCAAGTTGAAGCAATTTCTTAAAATGAATATCAGTGAAGAATTCAAT |
| | GTCCACTTACTAACAGTATCACAAGGCACACAAACACTGGTGAACTGCACAAGTAAGGA |
| | AGAAAAAAACGTAAAGGAACAGAAAAAGAATGATGCATGTTTCCTAAAGAGACTACTG |
| | AGAGAAATAAAAACTTGTTGGAATAAAATTTTGAAGGGCAGTATATAA |
| hIL-7 | ATGTTCCATGTTTCTTTTAGGTATATCTTTGGACTTCCTCCCCTGATCCTTGTTCTGTTGCC |
| SEQ ID NO: 4 | AGTAGCATCATCTGATTGTGATATTGAAGGTAAAGATGGCAAACAATATGAGAGTGTTC |
| | TAATGGTCAGCATCGATCAATTATTGGACAGCATGAAAGAAATTGGTAGCAATTGCCTG |
| | AATAATGAATTTAACTTTTTTAAAAGACATATCTGTGATGCTAATAAGGAAGGTATGTTT |
| | TTATTCCGTGCTGCTCGCAAGTTGAGGCAATTTCTTAAAATGAATAGCACTGGTGATTTT |
| | GATCTCCACTTATTAAAAGTTTCAGAAGGCACAACAATACTGTTGAACTGCACTGGCCAG |
| | GTTAAAGGAAGAAAACCAGCTGCCCTGGGTGAAGCCCAACCAACAAAGAGTTTGGAAG |
| | AAAATAAATCTTTAAAGGAACAGAAAAAACTGAATGACTTGTGTTTCCTAAAGAGACTA |
| | TTACAAGAGATAAAAACTTGTTGGAATAAAATTTTGATGGGCACTAAAGAACACTGA |

### D. FEATURES OF IMPLANTABLE CONSTRUCTS

The implantable construct described herein may take any suitable shape or morphology. For example, an implantable construct may be a sphere, spheroid, tube, cord, string, ellipsoid, disk, cylinder, sheet, torus, cube, stadiumoid, cone, pyramid, triangle, rectangle, square, or rod. An implantable construct may comprise a curved or flat section. In an embodiment, an implantable construct may be prepared through the use of a mold, resulting in a custom shape.

The implantable construct may vary in size, depending, for example, on the use or site of implantation. For example, an implantable construct may have a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a section or region with a mean diameter or size greater than 0.1 mm, e.g., greater than 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, or more. In an embodiment, an implantable construct may have a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller. In an embodiment, an implantable construct may have a section or region with a mean diameter or size less than 1 cm, e.g., less 50 mm, 40 mm, 30 mm, 20 mm, 10 mm, 7.5 mm, 5 mm, 2.5 mm, 1 mm, 0.5 mm, or smaller.

An implantable construct comprises at least one zone, which can be capable of preventing exposure of an enclosed antigen molecule or immune effector molecule from the outside milieu, e.g., a host effector cell or tissue. In an embodiment, the implantable construct comprises a layer or zone encapsulating or is admixed with the antigen molecule or immune effector molecule. In some embodiments, the construct comprises an inner zone (IZ). In an embodiment, the implantable construct comprises an outer zone (OZ). In an embodiment, either the inner zone (IZ) or outer zone (OZ) may be erodible or degradable. In an embodiment, either the inner zone (IZ) or outer zone (OZ) is not erodible or degradable. In an embodiment, the inner zone (IZ) is erodible or degradable. In an embodiment, the inner zone (IZ) is not readily erodible or degradable. In an embodiment, the outer zone (OZ) is erodible or degradable. In an embodiment, the outer zone (OZ) is not readily erodible or degradable. In an embodiment, the implantable construct comprises both an inner zone (IZ) and an outer zone (OZ), either of which may be erodible or degradable. In an embodiment, the implantable construct comprises both an inner zone (IZ) and an outer zone (OZ), wherein the outer zone is erodible or degradable. In an embodiment, the implantable construct comprises both an inner zone (IZ) and an outer zone (OZ), wherein the inner zone is erodible or degradable. The thickness of either of the zone, e.g., either the inner zone or outer zone, may be correlated with the length or duration of a “shielded” phase, in which the encapsulated antigen molecule or immune effector molecule is protected or shielded from the outside milieu, e.g., a host effector cell or tissue. In some embodiments, the construct comprises a single layer or zone that encapsulates or is admixed with the cell or other molecules provided for herein.

The zone (e.g., layer, the inner zone or outer zone) of the implantable construct may comprise a degradable entity, e.g., an entity capable of degradation. A degradable entity may comprise an enzyme cleavage site, a photolabile site, a pH-sensitive site, or other labile region that can be eroded or comprised over time. In an embodiment, the degradable entity is preferentially degraded upon exposure to a first condition (e.g., exposure to a first milieu, e.g., a first pH or first enzyme) relative to a second condition (e.g., exposure to a second milieu, e.g., a second pH or second enzyme). In one embodiment, the degradable entity is degraded at least 2, 5, 10, 20, 30, 40, 50, 60, 70, 80, or 100 times faster upon exposure to a first condition relative to a second condition. In an embodiment, the degradable entity is an enzyme cleavage site, e.g., a proteolytic site. In an embodiment, the degradable entity is a polymer (e.g., a synthetic polymer or a naturally occurring polymer, e.g., a peptide or polysaccharide). In an embodiment, the degradable entity is a substrate for an endogenous host component, e.g., a degradative enzyme, e.g., a remodeling enzyme, e.g., a collagenase or metalloprotease. In an embodiment, the degradable entity comprises a cleavable linker or cleavable segment embedded in a polymer.

In an embodiment, an implantable construct comprises a pore or opening to permit passage of an object, such as a small molecule (e.g., nutrients or waste), a protein, or a nucleic acid. For example, a pore in or on an implantable construct may be greater than 0.1 nm and less than 10 μm. In an embodiment, the implantable construct comprises a pore or opening with a size range of 0.1 μm to 10 μm, 0.1 μm to 9 μm, 0.1 μm to 8 μm, 0.1 μm to 7 μm, 0.1 μm to 6 μm, 0.1 μm to 5 μm, 0.1 μm to 4 μm, 0.1 μm to 3 μm, 0.1 μm to 2 μm.

An implantable construct described herein may comprise a chemical modification in or on any enclosed material. Exemplary chemical modifications include small molecules, peptides, proteins, nucleic acids, lipids, or oligosaccharides. The implantable construct may comprise at least 0.5%, 1%, 2%, 3%, 4%, 5%, 7.5%, 10%, 15%, 20%, 30%, 40%, 50%, 60%, 70%, 80% or more of a material that is chemically modified, e.g., with a chemical modification described herein. An implantable construct may be partially coated with a chemical modification, e.g., at least about 5%, 10%, 15%, 20%, 25%, 30%, 35%, 40%, 45%, 50%, 55%, 60%, 65%, 70%, 75%, 80%, 85%, 90%, 95%, 99%, or 99.9% coated with a chemical modification.

In an embodiment, the implantable construct is formulated such that the duration of release of the antigen molecule and/or immune effector molecule is tunable. For example, an implantable construct may be configured in a certain manner to release a specific amount of an antigen molecule and/or immune effector molecule over time, e.g., in a sustained or controlled manner. In an embodiment, the implantable construct comprises a zone (e.g., an inner zone or an outer zone) that is degradable, and this controls the duration of release from the construct by gradually ceasing immunoprotection of encapsulated cells or causing gradual release of the antigen molecule and/or immune effector molecule. In an embodiment, the implantable construct is configured such that the level of release of an antigen molecule and/or immune effector molecule is sufficient to modulate the ratio of a host effector cell, e.g., a host T cell. In an embodiment, the implantable construct is configured such that the level of release of an antigen molecule and/or immune effector molecule is sufficient to activate a host cell (e.g., a host T effector cell or a host NK cell) or increase the level of certain host cells (e.g., host T effector cells or host NK cells). In an embodiment, the implantable construct is configured such that the level of release of an antigen molecule and/or immune effector molecule is not sufficient to activate a host regulator cell (e.g., a host T regulator cell) or increase the level of host regulator cells (e.g., host T regulator cells).

In some embodiments, the implantable construct comprises a zone that is targeted by the natural foreign body response (FBR) of a host or subject, e.g., over a period of time. In an embodiment, the implantable construct is coated with fibrotic overgrowth upon administration to a subject, e.g., over a period of time. Fibrotic overgrowth on the surface of the implantable construct may lead to a decrease in function of the implantable construct. For example, a decrease in function may comprise a reduction in the release of an antigenic or therapeutic agent over time, a decrease in pore size, or a decrease in the diffusion rate of oxygen and other key nutrients to the encapsulated cells, leading to cell death. In an embodiment, the rate of fibrotic overgrowth may be tuned to design a dosing regimen. For example, the fibrotic overgrowth on the surface of an implantable construct may result in a decrease in function of the implantable construct about 6 hours, 12 hours, 18 hours, 1 day, 2 days, 3 days, 4 days, 5 days, 6 days, 7 days, 8 days, 9 days, 10 days, 11 days, 12 days, 13 days, 2 weeks, 2.5 weeks, 3 weeks, 4 weeks, or 6 weeks after administration (e.g., injection or implantation) to a subject.

In some embodiments, the implantable construct is chemically modified with a specific density of modifications. The specific density of chemical modifications may be described as the average number of attached chemical modifications per given area. For example, the density of a chemical modification on or in an implantable construct may be 0.01, 0.1, 0.5, 1, 5, 10, 15, 20, 50, 75, 100, 200, 400, 500, 750, 1,000, 2,500, or 5,000 chemical modifications per square μm or square mm.

An implantable construct may be formulated or configured for implantation in any organ, tissue, cell, or part of a subject. For example, the implantable construct may be implanted or disposed into the intraperitoneal space of a subject. An implantable construct may be implanted in or disposed on a tumor or other growth in a subject, or be implanted in or disposed about 0.1 mm, 0.5 mm, 1 mm, 0.25 mm, 0.5 mm, 0.75, 1 mm, 1.5 mm, 2 mm, 3 mm, 4 mm, 5 mm, 6 mm, 7 mm, 8 mm, 9 mm, 10 mm, 20 mm, 30 mm, 40 mm, 50 mm, 1 cm, 5, cm, 10 cm, or further from a tumor or other growth in a subject. An implantable construct may be configured for implantation, or implanted, or disposed on or in the skin, a mucosal surface, a body cavity, the central nervous system (e.g., the brain or spinal cord), an organ (e.g., the heart, eye, liver, kidney, spleen, lung, ovary, breast, uterus), the lymphatic system, vasculature, oral cavity, nasal cavity, gastrointestinal tract, bone, muscle, adipose tissue, skin, or other area.

An implantable construct may be formulated for use for any period of time. For example, an implantable construct may be used for 1 hour, 2 hours, 4 hours, 6 hours, 12 hours, 1 day, 36 hours, 2 days, 3 days, 4 days, 5 days, 6 days, 1 week, 2 weeks, 3 weeks, 4 weeks, 5 weeks, 6 weeks, 2 months, 3 months, 4 months, 5 months, 6 months, 1 year, or longer. An implantable construct can be configured for limited exposure (e.g., less than 2 days, e.g., less than 2 days, 1 day, 24 hours, 20 hours, 16 hours, 12 hours, 10 hours, 8 hours, 6 hours, 5 hours, 4 hours, 3 hours, 2 hours, 1 hour or less). A implantable construct can be configured for prolonged exposure (e.g., at least 2 days, 3 days, 4 days, 5 days, 6 days, 7 days, 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more). An implantable construct can be configured for permanent exposure (e.g., at least 6 months, 7 months, 8 months, 9 months, 10 months, 11 months, 12 months, 13 months, 14 months, 15 months, 16 months, 17 months, 18 months, 19 months, 20 months, 21 months, 22 months, 23 months, 24 months, 1 year, 1.5 years, 2 years, 2.5 years, 3 years, 3.5 years, 4 years or more).

### E. METHODS OF TREATMENT

Described herein are implantable constructs comprising a zone (e.g., a layer) encapsulating an antigen molecule and an immune effector molecule, and related methods of use thereof. In an embodiment, a composition of implantable constructs is used as a vaccine to protect a subject from an immune challenge, e.g., from a disease, e.g., as described herein. In an embodiment, a composition of implantable constructs is used to treat a disease, e.g., a disease described herein.

In some embodiments, the disease is a proliferative disease. In an embodiment, the proliferative disease is cancer. A cancer may be an epithelial, mesenchymal, or hematological malignancy. A cancer includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor). In an embodiment, the cancer is a solid tumor (e.g., carcinoid, carcinoma or sarcoma), a soft tissue tumor (e.g., a heme malignancy), or a metastatic lesion, e.g., a metastatic lesion of any of the cancers disclosed herein. In an embodiment, the cancer is a fibrotic or desmoplastic solid tumor.

Exemplary cancers include carcinoma, lymphoma, blastoma, sarcoma, and leukemia or lymphoid malignancies. In an embodiment, the cancer affects a system of the body, e.g., the nervous system (e.g., peripheral nervous system (PNS) or central nervous system (CNS)), vascular system, skeletal system, respiratory system, endocrine system, lymph system, reproductive system, or gastrointestinal tract. In some embodiments, cancer affects a part of the body, e.g., blood, eye, brain, skin, lung, stomach, mouth, ear, leg, foot, hand, liver, heart, kidney, bone, pancreas, spleen, large intestine, small intestine, spinal cord, muscle, ovary, uterus, vagina, or penis. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Other examples of cancers include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

In some embodiment, the disease is an infectious disease (e.g., a disease caused by a pathogen, e.g. a virus, bacterium, protozoa, or fungus). Exemplary infectious diseases include mumps; measles; rubella; rabies; bronchitis; polio; chicken pox; shingles; hepatitis A; hepatitis B; hepatitis C; hepatitis D; herpes simplex; acute immunodeficiency syndrome (AIDS); severe acute respiratory syndrome (SARS); severe acute respiratory syndrome coronavirus 2 (SARS CoV-2); respiratory syncytial infection; influenza; Ebola fever; Hanta fever; dengue fever; Reye syndrome; gonorrhea; norovirus infection; roseola; infectious mononucleosis; croup; hand, foot, and mouth disease; myocarditis, pericarditis; aseptic meningitis; bacterial meningitis; viral meningitis; bacterial vaginosis; cystitis; pneumonia; pharyngitis; keraconjunctivitis; gastroenteritis; warts; cold sores; botulism; bubonic plague; Chlamydia; cholera; Creutzfeldt-Jakob disease; typhus; giardiasis; Helicobacter pylori infection; hemorrhagic fever; hookworm; Epstein-Barr virus infectious mononucleosis; kuru; leprosy; Leishmaniasis; lassa fever; keratitis; Kawasaki disease; malaria; murine typhus; monkeypox; rhinosporidiosis; sepsis; scabies; rotavirus infection; tetanus; tinea cruris; tinea pedis; toxoplasmosis; typhoid fever; tularemia; trichomoniasis; Zika fever; yersiniosis; and West Nile fever.

In some embodiments, the disease implantable is neurodegenerative disease, autoimmune disease (e.g., diabetes, multiple sclerosis, lupus, occlusions, capsular contractions), or a liver disease (e.g., hepatitis B infection, hepatitis C infection, cirrhosis, or liver cancer). In some embodiments, the disease is diabetes (e.g., type 1 diabetes or type 2 diabetes). In some embodiments, the condition is fibrosis. In some embodiments, the condition is inflammation.

The implantable construct described herein may be used in a method to modulate (e.g., upregulate) the immune response in a subject. For example, upon administration to a subject, the implantable construct (or an antigen molecule or immune effector molecule disposed within) may modulate (e.g., upregulate) the level of a component of the immune system in a subject (e.g., increasing the level or decreasing the level of a component). Exemplary immune system components that may be modulated by a method described herein include T cells (e.g., an invasive T cell, a killer T cell, an effector T cell, a memory T cell, a gamma delta T cell, a helper T cell), B cells, antibodies, or other another component.

The implantable constructs described herein may further comprise an additional pharmaceutical agent, such as an anti-proliferative agent, anti-cancer agent, anti-inflammatory agent, an immunomodulatory agent, or a pain-relieving agent, e.g., for use in combination therapy. The additional pharmaceutical agent may be disposed in or on the implantable construct or may be produced by a cell disposed in or on the implantable construct. In an embodiment, the additional pharmaceutical agent is small molecule, a protein, a peptide, a nucleic acid, an oligosaccharide, or other agent.

In an embodiment, the additional pharmaceutical agent is an anti-cancer agent. In some embodiments, the anti-cancer agent is a small molecule, a kinase inhibitor, an alkylating agent, a vascular disrupting agent, a microtubule targeting agent, a mitotic inhibitor, a topoisomerase inhibitor, an anti-angiogenic agent, or an anti-metabolite. In an embodiment, the anti-cancer agent is a taxane (e.g., paclitaxel, docetaxel, larotaxel or cabazitaxel). In an embodiment, the anti-cancer agent is an anthracycline (e.g., doxorubicin). In some embodiments, the anti-cancer agent is a platinum-based agent (e.g., cisplatin or oxaliplatin). In some embodiments, the anti-cancer agent is a pyrimidine analog (e.g., gemcitabine). In some embodiments, the anti-cancer agent is chosen from camptothecin, irinotecan, rapamycin, FK506, 5-FU, leucovorin, or a combination thereof. In other embodiments, the anti-cancer agent is a protein biologic (e.g., an antibody molecule), or a nucleic acid therapy (e.g., an antisense or inhibitory double stranded RNA molecule).

In an embodiment, the additional pharmaceutical agent is an immunomodulatory agent, e.g., one or more of an activator of a costimulatory molecule, an inhibitor of an immune checkpoint molecule, or an anti-inflammatory agent. In an embodiment, the immunomodulatory agent is an inhibitor of an immune checkpoint molecule (e.g., an inhibitor of PD-1, PD-L1, LAG-3, TIM-3 or CTLA4, or any combination thereof). In some embodiments, the immunomodulatory agent is a cancer vaccine.

In some embodiments, the immunomodulatory agent is an inhibitor of PD-1, PD-L1, PD-L2, CTLA4, TIM3, LAG3, VISTA, BTLA, TIGIT, LAIR1, CD73, CD160, 2B4 and/or TGFR beta. In one embodiment, the inhibitor of an immune checkpoint molecule inhibits PD-1, PD-L1, LAG-3, TIM-3 or CTLA4, or any combination thereof. Inhibition of an inhibitory molecule can be performed at the DNA, RNA or protein level. In some embodiments, an inhibitory nucleic acid (e.g., a dsRNA, siRNA or shRNA), can be used to inhibit expression of an inhibitory molecule. In other embodiments, the inhibitor of an inhibitory signal is, a polypeptide e.g., a soluble ligand (e.g., PD-1-Ig or CTLA-4 Ig), or an antibody or antigen-binding fragment thereof, that binds to the inhibitory molecule; e.g., an antibody or fragment thereof that binds to PD-1, PD-L1, PD-L2, CTLA4, TIM3, LAG3, VISTA, BTLA, TIGIT, LAIR1, CD73, CD160, 2B4 and/or TGFR beta, or a combination thereof. In some embodiments, the immunomodulatory agent is an anti-inflammatory agent, e.g., an anti-inflammatory agent as described herein. In an embodiment, the anti-inflammatory agent is an agent that blocks, inhibits, or reduces inflammation or signaling from an inflammatory signaling pathway. In an embodiment, the anti-inflammatory agent inhibits or reduces the activity of one or more of any of the following an immune component of the subject. In an embodiment, the anti-inflammatory agent is an IL-1 or IL-1 receptor antagonist, such as anakinra, rilonacept, or canakinumab. In an embodiment, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In an embodiment, the anti-inflammatory agent is a TNF-α antagonist, e.g., an anti-TNF-α antibody, such as infliximab (REMICADE®), golimumab (SIMPONI®), adalimumab (HUMIRA®), certolizumab pegol (CIMZIA®) or etanercept. In one embodiment, the anti-inflammatory agent is a corticosteroid, e.g., as described herein.

### F. COMPOSITIONS AND ADMINISTRATIONS OF IMPLANTABLE CONSTRUCTS

The present disclosure features pharmaceutical compositions comprising implantable constructs comprising a zone (e.g., an inner zone and optionally an outer zone, both of which may be degradable), an antigen molecule, and immune effector molecule, and optionally a pharmaceutically acceptable excipient. In some embodiments, the implantable construct is provided in an effective amount in the pharmaceutical composition. In some embodiments, the effective amount is a therapeutically effective amount. In some embodiments, the effective amount is a prophylactically effective amount.

Pharmaceutical compositions described herein can be prepared by any method known in the art of pharmacology. In general, such preparatory methods include the steps of bringing the implantable construct into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the implantable construct may be generally equal to the dosage of the antigen molecule or immune effector molecule which would be administered to a subject and/or a convenient fraction of such a dosage such as, for example, one-half or one-third of such a dosage.

Relative amounts of the implantable construct, the pharmaceutically acceptable excipient, and/or any additional ingredients in a pharmaceutical composition of the disclosure will vary, depending upon the identity, size, and/or condition of the subject treated and further depending upon the route by which the composition is to be administered. By way of example, the composition may comprise between 0.1% and 100% (w/w) of any component.

The implantable construct and a pharmaceutical composition thereof may be administered or implanted orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally. In an embodiment, the implantable construct is injected subcutaneously. In an embodiment, the implantable construct is injected into the intraperitoneal space. In an embodiment, the implantable construct is injected into the intraperitoneal space. In an embodiment, the implantable constructed is delivered to the subject using a device, e.g., a cannula or catheter.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally or intravenously. Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For ophthalmic use, provided compounds, compositions, and devices may be formulated as micronized suspensions or in an ointment such as petrolatum.

In an embodiment, the release of an antigenic, therapeutic, or additional pharmaceutical agent is released in a sustained fashion. In order to prolong the effect of a particular agent, it is often desirable to slow the absorption of the agent from injection. This can be accomplished by the use of a liquid suspension of crystalline or amorphous material with poor water solubility. The rate of absorption of the agent then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

The implantable constructs provided herein are typically formulated in dosage unit form, e.g., single unit dosage form, for ease of administration and uniformity of dosage. It will be understood, however, that the total daily usage of the compositions of the present disclosure will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific therapeutic agent employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

An effective amount of an antigen molecule or immune effector molecule released from the implantable construct may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of therapeutic agent per unit dosage form (e.g, per implantable construct).

The antigen molecule or immune effector molecule administered may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

In addition, in some embodiments, the following embodiments are provided:
1. A composition comprising:
   a first implantable construct comprising an engineered cell that produces an antigen molecule; and
   a second implantable construct comprising an encapsulated engineered cell that produces an immune effector molecule.
2. The composition of embodiment 1, wherein the antigen molecule induces an immune response in a subject.
3. The composition of any one of embodiments 1-2, wherein the antigen molecule comprises a nucleic acid, a protein, an antibody, antibody fragment, enzyme, cytokine, hormone, receptor, a lipid, a small molecule, a metabolic agent, an oligosaccharide, a peptide, or an amino acid.
4. The composition of any one of embodiments 1-3, wherein the immune effector molecule activates an immune cell in a subject, represses an immune cell in a subject, and/or modulates immune cell migration in a subject.
5. The composition of any one of embodiments 1-4, wherein the immune effector molecule enhances an immune response in a subject.
6. The composition of any of embodiments 1-5, wherein the immune effector molecule modulates host dendritic cell migration and/or host T cell activation
7. The composition of any one of embodiments 1-6, wherein the immune effector molecule enhances the immune response to the antigen molecule.
8. The composition of embodiment 7, wherein the enhancing is specific for the antigen molecule.
9. The composition of any one of embodiments 1-8, wherein the antigen comprises an exogenous antigen, endogenous antigen, autoantigen, neoantigen, viral antigen, or tumor antigen.
10. The composition of any one of embodiments 1-9, wherein the immune effector molecule comprises a cytokine.
11. The composition of embodiment 10, wherein the cytokine is selected from IL-2, IL-12, IL-1, IL-1α, IL-1β, IL-1RA, IL-4, IL-5, IL-6, IL-7, IL-8, IL-9, IL-10, IL-11, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, and OSM.
12. The composition of embodiment 10, wherein the cytokine is selected from IL-2, IL-12a, IL-12b, IL-4, IL-7, IL-10, and IL-17.
13. The composition of any one of embodiments 10-12, wherein the sequence of the cytokine is as shown in Table 1.
14. The composition of any one of embodiments 1-13, wherein either the first implantable construct, the second implantable construct, or both comprise an layer or zone encapsulating or admixed with the engineered cell of the first or second construct
15. The composition of any one of embodiments 1-13, wherein either the first implantable construct, the second implantable construct, or both comprise an inner zone and/or an outer zone.
16. The composition of embodiment 15, wherein the outer zone is configured so as to hinder contact of a host immune effector molecule or cell with the inner zone for an initial or shielded phase of implantation, but allows contact of a host immune effector molecule or cell with the inner zone in a subsequent or unshielded phase of implantation.
17. The composition of embodiment 16, wherein the shielded phase lasts for no longer than 1 hour, 12 hours, 1 day, 2 days, 3 days, 6 days, or 12 days.
18. The composition of embodiment 16, wherein the shielded phase lasts for between 0.5 days and 30 days, 1 day and 14 days, and 1 day and 7 days.
19. The composition of any one of embodiments 15-18, wherein the thickness of the outer zone correlates with the length/duration of the shielded phase.
20. The composition of any one of embodiments 15-19, wherein erosion of the outer zone allows contact of host immune effector cells or molecules with the inner zone.
21. The composition of any of embodiments 14-20, wherein a reduction of the thickness of the layer, zone or outer zone is mediated by modification.
22. The composition of any of embodiments 14-20, wherein the layer, zone, or outer zone comprises a degradable entity comprising an enzyme cleavage site.
23. The composition of embodiment 22, wherein the degradable entity is cleaved by an endogenous host component.
24. The composition of 22 or 23, wherein the degradable entity is modulated by an exogenously applied treatment, e.g., the administration of an exogenous substance, treatment by the application of energy, e.g., heat or light, e.g., wherein modulation of the maintenance compound is mediated by temperature or light
25. The composition of any one of embodiments 1-24, wherein either the first implantable construct, the second implantable construct, or both comprise a polymer.
26. The composition of embodiment 25, wherein the polymer is alginate.
27. The composition of any one of embodiments 1-24, wherein both the first implantable construct and the second implantable construct comprise a polymer.
28. The composition of embodiment 27, wherein the polymer is alginate.
29. The composition of any one of embodiments 25-28, wherein the polymer is a modified alginate.
30. The composition of any one of embodiments 1-29, wherein the polymer is chemically modified, wherein the chemical modification can be to induce degradation of the polymer.
31. The composition of any one of embodiments 1-30, wherein the first implantable construct and the second implantable construct comprise the same polymer.
32. The composition of embodiment 31, wherein the polymer is alginate.
33. The composition of any one of embodiments 1-32, wherein the first implantable construct and the second implantable construct comprise different polymers.
34. The composition of any one of embodiments 1-33, wherein the first implantable construct provides sustained release of the antigen molecule.
35. The composition of any one of embodiments 1-34, wherein the first implantable construct provides substantially non-pulsatile release of the antigen molecule.
36. The composition of any one of embodiments 1-35, wherein the first implantable construct provides release of the antigen molecule for at least 1 day (e.g., longer than 2 days, 3 days, 4 days, 5 days, 6 days, 7 days, 8 days, 9 days, 10 days, 12 days, 14 days, 16 days, 18 days, or 20 days).
37. The composition of any of embodiments 1-36, wherein the first implantable construct provides release of the antigen molecule for at least 5 days (e.g., longer than 6 days, 7 days, 8 days, 9 days, 10 days, 12 days, 14 days, 16 days, 18 days, or 20 days).
38. The composition of any one of embodiments 1-37, wherein the second implantable construct provides sustained release of the immune effector molecule.
39. The composition of any one of embodiments 1-38, wherein the second implantable construct provides substantially non-pulsatile release of the immune effector molecule.
40. The composition of any one of embodiments 1-39, wherein the second implantable construct provides release of the immune effector cell for at least 1 day (e.g., longer than 2 days, 3 days, 4 days, 5 days, 6 days, 7 days, 8 days, 9 days, 10 days, 12 days, 14 days, 16 days, 18 days, or 20 days).
41. The composition of any one of embodiments 1-40, wherein the second implantable construct provides release of the immune effector cell for at least 5 days (e.g., longer than 6 days, 7 days, 8 days, 9 days, 10 days, 12 days, 14 days, 16 days, 18 days, or 20 days).
42. The composition of any one of embodiments 1-41, further comprising a third implantable element comprising an engineered cell that produces a second immune effector molecule.
43. A composition comprising:
   a first implantable construct comprising an engineered cell that produces an antigen molecule;
   a second implantable construct comprising an encapsulated engineered cell that produces an immune effector molecule (e.g., a cytokine); and
   a third implantable construct comprising an engineered cell that produces an immune effector molecule (e.g., a cytokine);
   wherein each implantable construct comprises, a layer, or zone encapsulating or admixed with the engineered cells or an inner zone and an outer zones, and
   wherein the layer, zone, or outer zone is configured so as to hinder contact of a host immune effector molecule or cell with the inner zone for an initial or shielded phase of implantation, but allows contact of a host immune effector molecule or cell with the inner zone in a subsequent or unshielded phase of implantation.
44. A method of forming a local or site-specific immune environment comprising implanting into a subject, the composition of any of embodiments 1-42.
45. A method of enhancing the immune response of a subject, comprising implanting into the subject, the composition of any of embodiments 1-42.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### G. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1: Encapsulation of Cells

Polyclonal ARPE-19 cells were expanded and transfected using a lipofectamine protocol with a ratio of 5:1 (transposase:transposon) to create cells expressing human native IL-12. Transfected cells were cultured are plated at 0.5 cells/well for single cell outgrowth. IL-12 production of the selected clone was about ˜3.8 PCD (picograms/cell/day). The clone was expanded in cell flasks/stacks for up to two weeks before being harvested into a cell pellet and suspended in alginate (SLG20) for encapsulation. The encapsulation process comprises loading two syringes, one with SLG20, and one with the cell pellet (42 million cells/mL) suspended in alginate (SLG20). The syringes were fed into a coaxial needle through use of a power supply (electric current) allowing droplets to fall into a crosslinking bath, which contained mannitol, barium chloride, HEPES buffer and Tween 20, which was where the capsules take shape. Capsules were collected from the bath after sitting in the bath for 5 minutes and washed 8 times at a 1:25 ratio of capsules to HEPES buffer solution (2 minutes/wash) to help to help remove loosely bound barium. The encapsulated cells were stored in DMEM/F12 cell culture media at ambient temperature in biotainer bottle.

### Example 2: Tumor Implantation Protocols

Implantable constructs will be investigated for their effects in vivo following the protocol described below.

Tumor Implants. The following mouse tumor models are prepared for implantation: (melanoma, Pan02, and ID8), and tracking of tumor size is carried out in vivo. C57BL6/J black mice are used for all in vivo tumor regression studies. To perform these studies, C57BL6/J black male and female mice (6-8 weeks old and each weighing ˜18-25 gm) are procured from Jackson Laboratories. All animal studies are performed based on the approval by IACUC approval.

Tumor Injection. For subcutaneous model of melanoma and Pan02, C57BL6/J mice are subcutaneously injected with 1 million cells (B16F10 or Pan02). The cells are suspended in saline and injected using an insulin syringe. For, IP tumor model for ID8 and Pan02, 5⁶ cells (suspended in saline) are intraperitoneally injected to the lower right abdomen of the C57BL6/J mice.

Subcutaneous Tumors. For SubQ melanoma and pancreatic tumor model melanoma, the tumor volume is measured by using the formula (0.5×ab 2), where ‘a’ represents the longest dimension and ‘b’ represents the shortest dimension of the tumors. Also, the weight and picture of the tumors are taken after the sacrifice.

Intraperitoneal Tumors. Animals injected with PAN02 or ID8 cancer cell lines are tracked every other day for 7-10 days before they are selected for implantation surgery, sham surgeries, or untreated control groups. After surgery, animals are tracked for tumor growth or reduction using small animal IVIS imaging (methods described below).

Animal Surgeries. Anesthesia & Analgesia: All procedures are conducted under anesthesia and treated with subsequent analgesia. Mice used in studies are anesthetized with isoflurane (1-4%) and oxygen (1-2 L) using a rodent anesthesia machine by tank induction followed by nose cone maintenance. Pedal withdrawal reflex is used to evaluate the depth of anesthesia for gas anesthesia before beginning any invasive procedure and respiration is monitored continuously to ensure sufficient anesthesia. Body temperatures are regulated during anesthesia and recovery through the use of water circulating heating pads. Importantly, the depth of anesthesia is continuously monitored throughout the surgeries.

Surgeon: Surgeons are required to wear a surgical mask and a clean lab coat. Sterile surgeon's gloves are used. When donning the sterile gloves, care is taken to prevent contamination of the outer sterile surface of the glove. Once gloved, care is taken to only touch the sterile operative field and equipment. Additional surgeon preparation precautions such as cap and sterile gown are employed when there are surgical cases with increased risk for clinical infection.

Intraperitoneal (IP) surgical implantation of capsules: Animals are all weighed, ear punch identified, and shaved along their abdomen prior to surgery as part of their pre-operative care. This is followed by one scrub with isopropyl alcohol followed by a second scrub with Betadine Surgical Scrub (7.5% povidone-iodine; Patterson Vet). These anti-septic scrubbing steps are repeated three times. A sharp surgical blade (15T; Sklar) is then used to cut a 0.5-0.75 cm midline incision through the skin and the linea alba into the abdomen. The surgeon keeps the incision as small as possible with 0.75 cm being the largest possible incision size. Capsule implants are introduced into the peritoneal cavity. The abdominal muscle is closed by suturing with 5-0 Ethicon black PDS-absorbable or other 5.0-6.0 monofilament absorbable sutures. The external skin layer is closed with PDS suture as previously described. Blood and tissue debris are removed from the surgical instruments between procedures with sterile water or saline and the instruments are resterilized between animals (maximum of 5 procedures) using a hot bead sterilizer. After the surgery, the animals are put back in the cage to recover on a heating pad and monitored until ambulating.

Post-Operative Care: Food is placed on the bottom of the cage for 48 hrs post-op to limit rearing and possible muscle suture rupture or hernias from recovering animals. Animals are monitored daily for four days post op for weight, general grooming, socialness, signs of dehiscence, dehydration, infection or blood loss from the suture site or in the stool. If suture sites are found open with no organ prolapse or significant bleeding the animals are anesthetized (1-4% isoflurane, 1-2 L/min O₂) in an induction tank and transferred to a nose cone for wound closure using wound glue (VetBond 3M, Patterson Vet).

Humane Endpoints & Euthanasia. if any adverse events were encountered during surgery animals are euthanized by anesthesia with 1-4% isoflurane & 1-2 L/min O₂ followed by 2 L/min CO₂ until signs of respiration are no longer evident. At this point, animals are cervically dislocated to ensure death. In the postoperative period, if humane endpoints are identified (lack of grooming, toe walking, hunched posture, social isolation, piloerected fur, severe dehiscence, significant blood loss) animals are euthanized. All euthanasia is in accordance with the approved IACUC protocol approved at Rice University and in accordance with the AVMA Guidelines for the Euthanasia of Animals.

| Cytokine Name | Sequences used |
|---|---|
| hIL-12 | SEQ ID NO: 5 |
| | ATGTGGCCCCCTGGGTCAGCCTCCCAGCCACCGCCCTCACCTGCCGCGGCCACAGGTCT |
| | GCATCCAGCGGCTCGCCCTGTGTCCCTGCAGTGCCGGCTCAGCATGTGTCCAGCGCGCA |
| | GCCTCCTCCTTGTGGCTACCCTGGTCCTCCTGGACCACCTCAGTTTGGCCAGAAACCTCC |
| | CCGTGGCCACTCCAGACCCAGGAATGTTCCCATGCCTTCACCACTCCCAAAACCTGCTGA |
| | GGGCCGTCAGCAACATGCTCCAGAAGGCCAGACAAACTCTAGAATTTTACCCTTGCACTT |
| | CTGAAGAGATTGATCATGAAGATATCACAAAAGATAAAACCAGCACAGTGGAGGCCTG |
| | TTTACCATTGGAATTAACCAAGAATGAGAGTTGCCTAAATTCCAGAGAGACCTCTTTCAT |
| | AACTAATGGGAGTTGCCTGGCCTCCAGAAAGACCTCTTTTATGATGGCCCTGTGCCTTAG |
| | TAGTATTTATGAAGACTTGAAGATGTACCAGGTGGAGTTCAAGACCATGAATGCAAAGC |
| | TTCTGATGGATCCTAAGAGGCAGATCTTTCTAGATCAAAACATGCTGGCAGTTATTGATG |
| | AGCTGATGCAGGCCCTGAATTTCAACAGTGAGACTGTGCCACAAAAATCCTCCCTTGAA |
| | GAACCGGATTTTTATAAAACTAAAATCAAGCTCTGCATACTTCTTCATGCTTTCAGAATTC |
| | GGGCAGTGACTATTGATAGAGTGATGAGCTATCTGAATGCTTCCTAA |
| | SEQ ID NO: 6 |
| | ATGTGTCACCAGCAGTTGGTCATCTCTTGGTTTTCCCTGGTTTTTCTGGCATCTCCCCTCG |
| | TGGCCATATGGGAACTGAAGAAAGATGTTTATGTCGTAGAATTGGATTGGTATCCGGAT |
| | GCCCCTGGAGAAATGGTGGTCCTCACCTGTGACACCCCTGAAGAAGATGGTATCACCTG |
| | GACCTTGGACCAGAGCAGTGAGGTCTTAGGCTCTGGCAAAACCCTGACCATCCAAGTCA |
| | AAGAGTTTGGAGATGCTGGCCAGTACACCTGTCACAAAGGAGGCGAGGTTCTAAGCCA |
| | TTCGCTCCTGCTGCTTCACAAAAAGGAAGATGGAATTTGGTCCACTGATATTTTAAAGGA |
| | CCAGAAAGAACCCAAAAATAAGACCTTTCTAAGATGCGAGGCCAAGAATTATTCTGGAC |
| | GTTTCACCTGCTGGTGGCTGACGACAATCAGTACTGATTTGACATTCAGTGTCAAAAGCA |
| | GCAGAGGCTCTTCTGACCCCCAAGGGGTGACGTGCGGAGCTGCTACACTCTCTGCAGAG |
| | AGAGTCAGAGGGGACAACAAGGAGTATGAGTACTCAGTGGAGTGCCAGGAGGACAGT |
| | GCCTGCCCAGCTGCTGAGGAGAGTCTGCCCATTGAGGTCATGGTGGATGCCGTTCACAA |
| | GCTCAAGTATGAAAACTACACCAGCAGCTTCTTCATCAGGGACATCATCAAACCTGACCC |
| | ACCCAAGAACTTGCAGCTGAAGCCATTAAAGAATTCTCGGCAGGTGGAGGTCAGCTGG |
| | GAGTACCCTGACACCTGGAGTACTCCACATTCCTACTTCTCCCTGACATTCTGCGTTCAG |
| | GTCCAGGGCAAGAGCAAGAGAGAAAAGAAAGATAGAGTCTTCACGGACAAGACCTCA |
| | GCCACGGTCATCTGCCGCAAAAATGCCAGCATTAGCGTGCGGGCCCAGGACCGCTACTA |
| | TAGCTCATCTTGGAGCGAATGGGCATCTGTGCCCTGCAGTTAG |
| mIL-15 | ATGAAAATTTTGAAACCATATATGAGGAATACATCCATCTCGTGCTACTTGTGTTTCCTTC |
| SEQ ID NO: 7 | TAAACAGTCACTTTTTAACTGAGGCTGGCATTCATGTCTTCATTTTGGGCTGTGTCAGTGT |
| | AGGTCTCCCTAAAACAGAGGCCAACTGGATAGATGTAAGATATGACCTGGAGAAAATT |
| | GAAAGCCTTATTCAATCTATTCATATTGACACCACTTTATACACTGACAGTGACTTTCATC |
| | CCAGTTGCAAAGTTACTGCAATGAACTGCTTTCTCCTGGAATTGCAGGTTATTTTACATG |
| | AGTACAGTAACATGACTCTTAATGAAACAGTAAGAAACGTGCTCTACCTTGCAAACAGC |
| | ACTCTGTCTTCTAACAAGAATGTAGCAGAATCTGGCTGCAAGGAATGTGAGGAGCTGGA |
| | GGAGAAAACCTTCACAGAGTTTTTGCAAAGCTTTATACGCATTGTCCAAATGTTCATCAA |
| | CACGTCCTGA |

| Cytokine Name | Sequences used |
|---|---|
| hIL-15 | ATGAGAATTTCGAAACCACATTTGAGAAGTATTTCCATCCAGTGCTACTTGTGTTTACTTC |
| SEQ ID NO: 8 | TAAACAGTCATTTTCTAACTGAAGCTGGCATTCATGTCTTCATTTTGGGCTGTTTCAGTGC |
| | AGGGCTTCCTAAAACAGAAGCCAACTGGGTGAATGTAATAAGTGATTTGAAAAAAATTG |
| | AAGATCTTATTCAATCTATGCATATTGATGCTACTTTATATACGGAAAGTGATGTTCACCC |
| | CAGTTGCAAAGTAACAGCAATGAAGTGCTTTCTCTTGGAGTTACAAGTTATTTCACTTGA |
| | GTCCGGAGATGCAAGTATTCATGATACAGTAGAAAATCTGATCATCCTAGCAAACAACA |
| | GTTTGTCTTCTAATGGGAATGTAACAGAATCTGGATGCAAAGAATGTGAGGAACTGGAG |
| | GAAAAAAATATTAAAGAATTTTTGCAGAGTTTTGTACATATTGTCCAAATGTTCATCAAC |
| | ACTTCTTGA |
| Firefly | ATGGAAGACGCCAAAAACATAAAGAAAGGCCCGGCGCCATTCTATCCGCTAGAGGATG |
| Luciferase | GAACCGCTGGAGAGCAACTGCATAAGGCTATGAAGAGATACGCCCTGGTTCCTGGAAC |
| SEQ ID NO: 9 | AATTGCTTTTACAGATGCACATATCGAGGTGAACATCACGTACGCGGAATACTTCGAAAT |
| | GTCCGTTCGGTTGGCAGAAGCTATGAAACGATATGGGCTGAATACAAATCACAGAATCG |
| | TCGTATGCAGTGAAAACTCTCTTCAATTCTTTATGCCGGTGTTGGGCGCGTTATTTATCG |
| | GAGTTGCAGTTGCGCCCGCGAACGACATTTATAATGAACGTGAATTGCTCAACAGTATG |
| | AACATTTCGCAGCCTACCGTAGTGTTTGTTTCCAAAAAGGGGTTGCAAAAAATTTTGAAC |
| | GTGCAAAAAAAATTACCAATAATCCAGAAAATTATTATCATGGATTCTAAAACGGATTAC |
| | CAGGGATTTCAGTCGATGTACACGTTCGTCACATCTCATCTACCTCCCGGTTTTAATGAAT |
| | ACGATTTTGTACCAGAGTCCTTTGATCGTGACAAAACAATTGCACTGATAATGAACTCCT |
| | CTGGATCTACTGGGTTACCTAAGGGTGTGGCCCTTCCGCATAGAACTGCCTGCGTCAGA |
| | TTCTCGCATGCCAGAGATCCTATTTTTGGCAATCAAATCATTCCGGATACTGCGATTTTAA |
| | GTGTTGTTCCATTCCATCACGGTTTTGGAATGTTTACTACACTCGGATATTTGATATGTGG |
| | ATTTCGAGTCGTCTTAATGTATAGATTTGAAGAAGAGCTGTTTTTACGATCCCTTCAGGA |
| | TTACAAAATTCAAAGTGCGTTGCTAGTACCAACCCTATTTTCATTCTTCGCCAAAAGCACT |
| | CTGATTGACAAATACGATTTATCTAATTTACACGAAATTGCTTCTGGGGGCGCACCTCTT |
| | TCGAAAGAAGTCGGGGAAGCGGTTGCAAAACGCTTCCATCTTCCAGGGATACGACAAG |
| | GATATGGGCTCACTGAGACTACATCAGCTATTCTGATTACACCCGAGGGGGATGATAAA |
| | CCGGGCGCGGTCGGTAAAGTTGTTCCATTTTTTGAAGCGAAGGTTGTGGATCTGGATAC |
| | CGGGAAAACGCTGGGCGTTAATCAGAGAGGCGAATTATGTGTCAGAGGACCTATGATT |
| | ATGTCCGGTTATGTAAACAATCCGGAAGCGACCAACGCCTTGATTGACAAGGATGGATG |
| | GCTACATTCTGGAGACATAGCTTACTGGGACGAAGACGAACACTTCTTCATAGTTGACC |
| | GCTTGAAGTCTTTAATTAAATACAAAGGATACCAGGTGGCCCCCGCTGAATTGGAGTCG |
| | ATATTGTTACAACACCCCAACATCTTCGACGCGGGCGTGGCAGGTCTTCCCGACGATGA |
| | CGCCGGTGAACTTCCCGCCGCCGTTGTTGTTTTGGAGCACGGAAAGACGATGACGGAAA |
| | AAGAGATCGTGGATTACGTCGCCAGTCAAGTAACAACCGCGAAAAAGTTGCGCGGAGG |
| | AGTTGTGTTTGTGGACGAAGTACCGAAAGGTCTTACCGGAAAACTCGACGCAAGAAAA |
| | ATCAGAGAGATCCTCATAAAGGCCAAGAAGGGCGGAAAGTCCAAATTGTAA |
| Metridia | ATGGACATCAAGGTGGTGTTCACCCTGGTGTTCAGCGCCCTGGTGCAGGCCAAGAGCAC |
| Luciferase | CGAGTTCGACCCCAACATCGACATCGTGGGCCTGGAAGGCAAGTTCGGCATCACCAACC |
| SEQ ID NO: 10 | TGGAAACCGACCTGTTCACCATCTGGGAGACCATGGAAGTGATGATCAAGGCCGACATC |
| | GCCGACACCGACCGGGCCAGCAACTTCGTGGCCACCGAGACCGACGCCAACCGGGGCA |
| | AGATGCCCGGCAAGAAGCTGCCCCTGGCCGTCATCATGGAAATGGAAGCCAACGCCTTC |
| | AAGGCCGGCTGCACCCGGGGCTGCCTGATCTGCCTGAGCAAGATCAAGTGCACCGCCA |
| | AGATGAAGGTGTACATCCCCGGCAGGTGCCACGACTACGGCGGCGACAAGAAAACCGG |
| | CCAGGCCGGCATCGTGGGCGCCATCGTGGACATCCCCGAGATCAGCGGCTTCAAAGAA |
| | ATGGCCCCCATGGAACAGTTCATCGCCCAGGTGGACAGATGCGCCAGCTGCACCACCGG |
| | CTGCCTGAAGGGCCTGGCCAACGTGAAGTGCAGCGAGCTGCTGAAGAAGTGGCTGCCC |
| | GACCGCTGCGCCAGCTTCGCCGACAAGATCCAGAAAGAGGTGCACAACATCAAGGGCA |
| | TGGCCGGCGACAGGTGA |

### Example 3: Subcutaneous or Intraperitoneal Administration of RPE-mIL12 Delays Subcutaneous Tumor Growth in B16F10 Melanoma Model

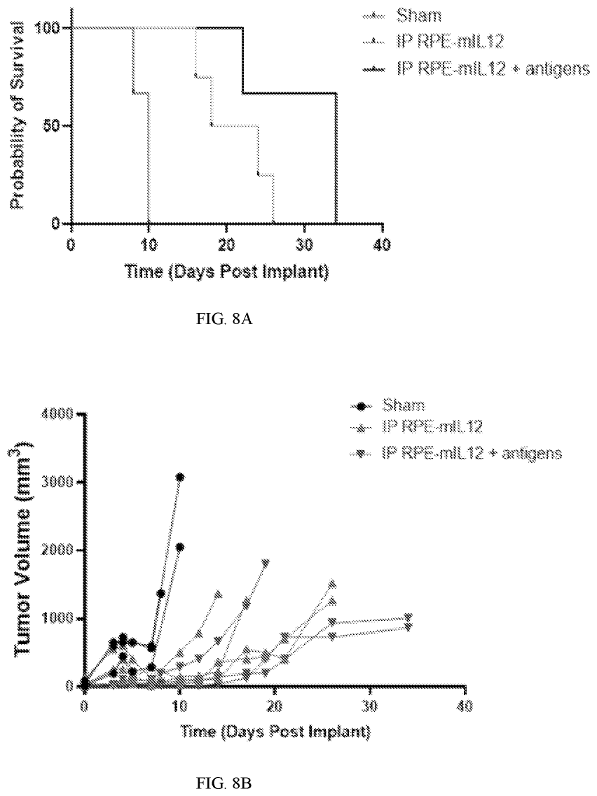

B16F10 (5×10⁵) cells suspended in HBSS were injected subcutaneously into the right flank B6 mice (mixed gender). 6 days after IP injections, tumors were measured using a digital caliper and stratified into groups of 3-4 mice. 7 days after IP injections, mice were treated with: 1) sham surgery, 2) 10 RPE-mIL12 capsules implanted in IP cavity, or 3) 10 RPE-mIL12 capsules+5 freeze/thawed B16F10 capsules implanted in the IP cavity. All capsules were administered via surgical implantation. The “antigens” were prepared according to the following: B16F10 melanoma cells were encapsulated into alginate capsules and subjected to 3 rounds of freezing/thawing cycles at −80° C. to disrupt the cells. At the time of administration, antigen capsules were thawed and administered along with 10 RPE-mIL12 capsules in the IP space of mice bearing subcutaneous B16F10 tumors. After capsule administration, tumors were measured every 2-4 days until tumors reached 15 mm in any direction. Treatment with RPE-mIL12 capsules or RPE-mIL12 capsules co-administered with antigens increased survival and delayed tumor growth, as illustrated in FIG. 8A and FIG. 8B, respectively.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

All publications, patent applications, patents, and other references mentioned herein are incorporated by reference in their entirety and for the purpose as context dictates.

The following listing of claims replaces all previous listings or versions thereof:

1. A composition comprising:
a first implantable construct comprising an encapsulated engineered cell that produces an antigen molecule; and
a second implantable construct comprising an encapsulated engineered cell that produces an immune effector molecule.

2. The composition of claim 1, wherein the antigen molecule induces an immune response in a subject.

3. The composition of claim 1, wherein the antigen molecule comprises a nucleic acid, a protein, an antibody, antibody fragment, enzyme, cytokine, hormone, receptor, a lipid, a small molecule, a metabolic agent, an oligosaccharide, a peptide, or an amino acid.

4. The composition of claim 1, wherein the immune effector molecule activates an immune cell in a subject, represses an immune cell in a subject, and/or modulates immune cell migration in a subject.

5. The composition of claim 1, wherein the immune effector molecule enhances an immune response in a subject, modulates host dendritic cell migration and/or host T cell activation, or enhances the immune response to the antigen molecule.

6-8. (canceled)

9. The composition of claim 1, wherein the antigen comprises an exogenous antigen, an endogenous antigen, an autoantigen, a neoantigen, a viral antigen, or a tumor antigen.

10. The composition of claim 1, wherein the immune effector molecule comprises a cytokine.

11. The composition of claim 10, wherein the cytokine is selected from IL-2, IL-12, IL-1, IL-1α, IL-1β, IL-1RA, IL-4, IL-5, IL-6, IL-7, IL-8, IL-9, IL-10, IL-11, IL-12a, IL-12b, IL-13, IL-14, IL-16, IL-17, G-CSF, GM-CSF, IL-20, IFN-α, IFN-β, IFN-γ, CD154, LT-β, CD70, CD153, CD178, TRAIL, TNF-α, TNF-β, SCF, M-CSF, MSP, 4-1BBL, LIF, and OSM.

12-13. (canceled)

14. The composition of claim 1, wherein either the first implantable construct and the second implantable construct each comprise a layer encapsulating the engineered cell of the first implantable construct and the second implantable construct.

15-24. (canceled)

25. The composition of claim 14, wherein the layer comprises a polymer.

26. The composition of claim 25, wherein the polymer is alginate.

27-33. (canceled)

34. The composition of claim 1, wherein the first implantable construct provides sustained release of the antigen molecule.

35. The composition of claim 1, wherein the first implantable construct provides substantially non-pulsatile release of the antigen molecule.

36. The composition of claim 1, wherein the first implantable construct provides release of the antigen molecule for at least 1 day at least 2 days, at least 3 days, at least 4 days, at least 5 days, at least 6 days, at least 7 days, at least 8 days, at least 9 days, at least 10 days, at least 12 days, at least 14 days, at least 16 days, at least 18 days, or at least 20 days.

37. (canceled)

38. The composition of claim 1, wherein the second implantable construct provides sustained release of the immune effector molecule.

39. The composition of claim 1, wherein the second implantable construct provides substantially non-pulsatile release of the immune effector molecule.

40. The composition of claim 1, wherein the second implantable construct provides release of the immune effector cell for at least 1 day at least 2 days, at least 3 days, at least 4 days, at least 5 days, at least 6 days, at least 7 days, at least 8 days, at least 9 days, at least 10 days, at least 12 days, at least 14 days, at least 16 days, at least 18 days, or at least 20 days.

41. (canceled)

42. The composition of claim 1, further comprising a third implantable element comprising an encapsulated engineered cell that produces a second immune effector molecule.

43. A composition comprising:
a first implantable construct comprising an encapsulated engineered cell that produces an antigen molecule;
a second implantable construct comprising an encapsulated engineered cell that produces an immune effector molecule; and
a third implantable construct comprising an engineered cell that produces an immune effector molecule;
wherein each implantable construct comprises a layer.

44. A method of forming a local or site-specific immune environment, or enhancing the immune response of a subject, comprising implanting into the subject, the composition of claim 1.

45. (canceled)


SEQUENCE LISTING (10 sequences)

SEQ ID NO: 1 - DNA, 510 bp, Artificial sequence
  Synthetic nucleic acid
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 2 - DNA, 462 bp, Artificial sequence
  Synthetic nucleic acid
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 3 - DNA, 465 bp, Artificial sequence
  Synthetic nucleic acid
atgttccatgtttcttttagatatatctttggaattcctccactgatccttgttctgctgcctgtcacatcatctgagtgccacattaaagacaaagaaggtaaagcatatgagagtgtactgatgatcagcatcgatgaattggacaaaatgacaggaactgatagtaattgcccgaataatgaaccaaacttttttagaaaacatgtatgtgatgatacaaaggaagctgcttttctaaatcgtgctgctcgcaagttgaagcaatttcttaaaatgaatatcagtgaagaattcaatgtccacttactaacagtatcacaaggcacacaaacactggtgaactgcacaagtaaggaagaaaaaaacgtaaaggaacagaaaaagaatgatgcatgtttcctaaagagactactgagagaaataaaaacttgttggaataaaattttgaagggcagtatataa

SEQ ID NO: 4 - DNA, 534 bp, Artificial sequence
  Synthetic nucleic acid
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacactga

SEQ ID NO: 5 - DNA, 762 bp, Artificial sequence
  Synthetic nucleic acid
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 6 - DNA, 987 bp, Artificial sequence
  Synthetic nucleic acid
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 7 - DNA, 489 bp, Artificial sequence
  Synthetic nucleic acid
atgaaaattttgaaaccatatatgaggaatacatccatctcgtgctacttgtgtttccttctaaacagtcactttttaactgaggctggcattcatgtcttcattttgggctgtgtcagtgtaggtctccctaaaacagaggccaactggatagatgtaagatatgacctggagaaaattgaaagccttattcaatctattcatattgacaccactttatacactgacagtgactttcatcccagttgcaaagttactgcaatgaactgctttctcctggaattgcaggttattttacatgagtacagtaacatgactcttaatgaaacagtaagaaacgtgctctaccttgcaaacagcactctgtcttctaacaagaatgtagcagaatctggctgcaaggaatgtgaggagctggaggagaaaaccttcacagagtttttgcaaagctttatacgcattgtccaaatgttcatcaacacgtcctga

SEQ ID NO: 8 - DNA, 489 bp, Artificial sequence
  Synthetic nucleic acid
atgagaatttcgaaaccacatttgagaagtatttccatccagtgctacttgtgtttacttctaaacagtcattttctaactgaagctggcattcatgtcttcattttgggctgtttcagtgcagggcttcctaaaacagaagccaactgggtgaatgtaataagtgatttgaaaaaaattgaagatcttattcaatctatgcatattgatgctactttatatacggaaagtgatgttcaccccagttgcaaagtaacagcaatgaagtgctttctcttggagttacaagttatttcacttgagtccggagatgcaagtattcatgatacagtagaaaatctgatcatcctagcaaacaacagtttgtcttctaatgggaatgtaacagaatctggatgcaaagaatgtgaggaactggaggaaaaaaatattaaagaatttttgcagagttttgtacatattgtccaaatgttcatcaacacttcttga

SEQ ID NO: 9 - DNA, 1653 bp, Artificial sequence
  Synthetic nucleic acid
atggaagacgccaaaaacataaagaaaggcccggcgccattctatccgctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaactcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggataccaggtggcccccgctgaattggagtcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaattgtaa

SEQ ID NO: 10 - DNA, 660 bp, Artificial sequence
  Synthetic nucleic acid
atggacatcaaggtggtgttcaccctggtgttcagcgccctggtgcaggccaagagcaccgagttcgaccccaacatcgacatcgtgggcctggaaggcaagttcggcatcaccaacctggaaaccgacctgttcaccatctgggagaccatggaagtgatgatcaaggccgacatcgccgacaccgaccgggccagcaacttcgtggccaccgagaccgacgccaaccggggcaagatgcccggcaagaagctgcccctggccgtcatcatggaaatggaagccaacgccttcaaggccggctgcacccggggctgcctgatctgcctgagcaagatcaagtgcaccgccaagatgaaggtgtacatccccggcaggtgccacgactacggcggcgacaagaaaaccggccaggccggcatcgtgggcgccatcgtggacatccccgagatcagcggcttcaaagaaatggcccccatggaacagttcatcgcccaggtggacagatgcgccagctgcaccaccggctgcctgaagggcctggccaacgtgaagtgcagcgagctgctgaagaagtggctgcccgaccgctgcgccagcttcgccgacaagatccagaaagaggtgcacaacatcaagggcatggccggcgacaggtga